(19) United States
(12) Patent Application Publication
(10) Pub. No.: US 2012/0245029 A1
(43) Pub. Date: Sep. 27, 2012

(54) NOVEL PHOSPH(ON)ATE- AND SULF(ON)ATE-BASED PHOSPHATE MODIFIED NUCLEOSIDES USEFUL AS SUBSTRATES FOR POLYMERASES AND AS ANTIVIRAL AGENTS

(75) Inventors: Piet Herdewijn, Wezemaal (BE); Philippe Marlière, Paris (FR)

(73) Assignee: Katholieke Universiteit Leuven, Leuven (BE)

(21) Appl. No.: 13/514,007

(22) PCT Filed: May 12, 2010
(86) PCT No.: PCT/EP2010/056599

§ 371 Date: Jun. 5, 2012

(30) Foreign Application Priority Data
Dec. 8, 2009 (GB) 0921443.8

(51) Int. Cl. C07H 19/20; A61P 31/12; A61P 31/18; C12N 7/00; A01P 21/00; C12N 1/16; C12N 1/14; C12N 1/12; A01N 57/32; C12N 5/00; A61K 31/7076; C12N 1/20

(52) U.S. Cl. 504/196; 536/26.7; 536/26.2; 514/47; 435/235.1; 435/252.1; 435/255.1; 435/254.1; 435/257.1; 435/325

(57) ABSTRACT

This invention provides phosphate-modified nucleosides represented by the structural formula (I): wherein W is O or S, and wherein B, R¹; R³ and R². are as defined herein. These compounds are useful as substrates for DNA/RNA polymerases, and as anti-viral agents in particular against HIV-1.

### FIELD OF THE INVENTION

The present invention relates to novel phosphate-modified nucleosides, such as carboxylic acid-, sulfate-, sulfonate-, phosphate- and/or phosphonate-containing phosphoramidate nucleosides. The present invention also relates to the phosphate-modified nucleosides as substrates for wild type and/or mutated DNA or RNA polymerases.

The present invention provides for the use of these novel phosphate-modified nucleosides for the production of oligonucleotides such as DNA or RNA and of polypeptides or proteins. The invention also relates to the use of these phosphate-modified nucleosides for growing or selecting specific micro-organisms, such as bacteria. The invention further provides for the use of these novel phosphate-modified nucleosides to treat or prevent viral infections and their use to manufacture a medicine to treat or prevent viral infections, particularly infections with viruses belonging to the HIV family. The present invention furthermore relates to a method for the production of oligonucleotides, peptides or proteins by using said phosphate-modified nucleosides.

### BACKGROUND OF THE INVENTION

There has been significant progress in the design and synthesis of numerous nucleotide analogues bearing a modified nucleobase moiety or unnatural sugar and that are substrates for polymerases. Modifications at the phosphate moiety are introduced to increase the stability of a nucleotide toward enzymatic degradation or to mask the phosphate negative charge and facilitate its penetration into a cell. Common strategy in nucleotide prodrug design is protecting a phosphate moiety with a labile masking group. Removal of a masking group liberates a nucleoside monophosphate entity to be transformed into a nucleoside triphosphate (hereinafter referred as NTP), a substrate for intracellular enzymes. However, even after removal of the masking group, phosphorylation and activation of nucleoside monophosphate remains a problem due to substrate specificity of cellular kinases. Therefore, design of a nucleotide analogue that would allow bypassing the kinase activation pathway while behaving as a direct polymerase substrate would be a considerable challenge.

Treatment of certain viral infections has always been a challenging task due to ability of some viruses to integrate into a host's genome. Therefore, the viral enzymes that are critical for viral genome replication and integration are regarded as the most effective targets for the design of anti-viral agents.

A lot of attention has been given to studying mechanisms of action of Human Immunodeficiency Virus (type 1) (HIV-1) and developing specific inhibitors towards this very challenging and important target. One of the enzymes that are essential for the HIV replication is HIV reverse transcriptase (HIV RT). The function of this enzyme is to use a viral RNA genome and a reverse transcriptase to synthesize a double stranded DNA for integration into a host genome. Because this step is critical for the propagation of the viral infection, HIV reverse transcriptase (RT) is an excellent target for anti-viral treatment. Currently, two major classes of RT inhibitors (RTIs) exist and are administered for treatment of HIV infection. Non-nucleoside reverse transcriptase inhibitors (NNRTs) are a group of compounds that act through the allosteric inhibition by binding to a hydrophobic site, or a pocket in close proximity to the active site of HIV RT. The other group of RTIs is represented by nucleoside reverse transcriptase inhibitors (NRTIs) that bind directly to the active site and interfere with the polymerization reaction and DNA synthesis.

Nucleoside reverse transcriptase inhibitors are designed to be recognized as substrates for RT and incorporated into a growing strand for further termination of chain elongation. Inhibition of reverse transcriptase activity and chain termination by NRTIs is achieved by introduction of structural modifications to the sugar moiety. The elongation of the DNA strand by a polymerase requires a nucleophilic attack of the 3′-OH group to the a phosphorus atom of an incoming nucleotide. Therefore, nucleoside analogs that lack the 3′-OH group or have it substituted with other functional groups (for instance, N₃, F, H) not capable of the nucleophilic attack and formation of phosphodiester bond would act as chain terminators.

Termination of DNA or RNA synthesis with nucleoside analogues is a common and one of the most efficient strategies in the treatment of viral infections, regardless of various side effects and cell toxicity. The therapeutically active form of a nucleoside analogue is a nucleoside triphosphate. However, at the physiological pH nucleoside triphosphates are negatively charged molecules and thus they can not penetrate cellular membranes. Hence, RT inhibitors are usually administered as biologically inactive free nucleosides or as monophosphate prodrugs where a phosphate group is masked with a lipophilic group.

There are three steps of kinase-mediated activation of anti-viral nucleosides. At first, transformation to a monophosphate derivative takes place through the action of a cytoplasmic nucleoside kinase (for instance, thymidine kinase and deoxycytidine kinase). Furthermore, a nucleoside 5′-monophosphate kinase catalyzes the conversion of a nucleoside monophosphate to a nucleoside diphosphate. Finally, a diphosphate derivative is phosphorylated by a nucleoside 5′-diphosphate kinase (NDK) to provide an anti-viral nucleoside analog in its activated (phosphorylated) form. The efficiency of phosphorylation depends on substrate specificity of kinases. For instance, in the case of the AZT phosphorylation cascade, conversion from the nucleoside monophosphate to the nucleoside diphosphate becomes a rate limiting step as thymidylate kinase (TMPK) catalyzes this conversion significantly slower than in the case of the natural substrate (TMP). The consequences of this inefficiency are accumulation of AZTMP in the cytosol and decreased therapeutic concentration of AZTTP, the activated nucleoside form.

However, it was determined that high levels of AZTMP have an inhibitory effect on thymidylate kinase by competing with its natural substrate (TMP) and resulting in reduced levels of TDP and TTP. Moreover, increased levels of AZT and its phosphorylated derivatives also affect other enzymes of the de novo dNTPs synthesis resulting in skewed natural nucleotide concentrations.

Therefore administration of free NRTIs, which often relies on intracellular phosphorylation and activation, has significant drawbacks. One of the possible solutions is a prodrug or pronucleotide approach. In the prodrug approach, the monophosphate moiety is “masked” with a labile functional group which also serves to facilitate passage of a “masked” nucleotide inside the cell. Once inside the cell, a masking group is removed either enzymatically or through chemical activation. Removal of the masking group affords a free nucleoside monophosphate intracellularly where it can be further phosphorylated by TMPK and NDK. Thus, although the prodrug approach facilitates delivery of an inhibitory nucleoside inside the cell and eliminates the need for initial phosphorylation by a nucleoside kinase, phosphorylation by TMPK and NDK are still required.

Besides delivery and bio-distribution challenges, another drawback that is often associated with anti-viral therapy is emergence of resistant strains. In the case of HIV-1, the drug resistance is developed by appearance of mutations that would allow HIV RT to discriminate NRTIs for natural nucleotides or remove an incorporated unnatural nucleobase by excision reactions. It has also been shown for herpes simplex virus (HSV) that reduction in anti-herpetic activity of acyclovir, a drug activated by thymidine kinase phosphorylation and commonly used for treatment of HSV infections, is mostly associated with thymidine kinase dependent resistance. Established strategies to manage acyclovir-resistant HSV infections include administration of anti-viral drugs acting directly on a viral DNA polymerase (foscarnet, cidifovir) or by modulating immune response of a patient. However, the later approach is not always feasible and the former one could worsen patient's condition since these medications impose a significant level of toxicity.

WO 00/08040 discloses cytidinyl phosphates, phosphoamidates and phosphorotiolates as having sialyltransferase inhibiting activity and therefore being useful for inhibiting intracellular adhesion and for treating certain inflammatory diseases. According to the broad structural formula of claim 1, a group X¹CR³R⁴R⁵ is attached to the phosphorus atom of these compounds wherein X¹ is selected from the group consisting of O, S, NH, CH₂ and CF₂, and wherein each of R³, R⁴ and R⁵ is independently selected from the group consisting of hydrogen, carboxyl, phosphonyl, sulfonyl, alkyl, aryl, alkylaryl and heteroaryl. FIGS. 3 and 6 disclose producing compounds listed in table 1 (an embodiment wherein X¹ is O, R³ is aryl or heteroaryl or heterocyclyl, one of R⁴ and R⁵ is hydrogen, and the other one of R⁴ and R⁵ is carboxyl or phosphonyl) in a two-steps synthetic procedure starting from (a) a dibenzyl hydroxy-(aryl)methylphosphonate and (b) 2′,3′-di-O-acetylcytidinyloxy)-cyanoethoxy-diisopropylaminophosphane. No general synthetic protocol is disclosed for making other compounds according to the broad structural formula, In particular, no procedure or starting material is disclosed for making phosphoramidates or phosphorothiolates. The disclosed compounds also do not include a nucleobase other than cytidine or a sugar moiety other than ribose. Their suggested medicinal uses are limited to the fields of inflammation and cancer.

WO 03/072757 discloses modified diphosphate nucleoside mimics wherein the 5′ position of said nucleoside is attached to a group represented by the structural formula

—P(O)(OH)—X⁵—P(O)X⁸X¹⁰

wherein

X⁵ may be NH or NR,

R is selected from the group consisting of alkyl, aryl and aralkyl, and

X⁸ and X¹⁰ may be OH,

and prodrugs thereof. Illustrative compounds 3, 6 and 8 of that kind are shown on page 61. Such modified diphosphate nucleoside mimics are useful for the inhibition of DNA and RNA polymerases, for the treatment of infectious diseases caused by viruses (e.g. HIV, HCV, etc), bacteriae and fungi, and for the treatment of prolifertive disorders. This document however does not teach any nucleoside or deoxynucleoside phosphates, phosphorothiolates or phosphoramidates including pending carboxylic acid groups or sulfonic acid groups.

WO 2007/020018 discloses the use of a modified nucleoside in combination with an antigen for inducing immunotolerance to said antigen. Among the illustrative nucleosides disclosed in table A (page 23) are compounds 13 and 15 wherein the group attached to the nucleoside is represented by the formula:

—P(O)(OH)—NH—P(O)(OH)₂.

This document does not disclose the preparation of these modified nucleosides, but this is known from WO 03/072757. The nucleosides as defined in claim 1 of this document, however do not include any aryl, carboxyl or sulfonyl groups. Their suggested medicinal use is limited to the field of immunization.

WO 2008/012555 discloses modified nucleosides for use in the treatment of a glycolipid-mediated autoimmune disease. The 5′ position of said nucleoside is attached to a group represented by the structural formula

—(CH₂)—P(O)(OH)R

i.e. without a P—O linkage. In addition this document discloses a few modified phosphate nucleosides wherein the phosphate group is attached via a P—O linkage, in particular compounds 46a-h, said to be described in Bioorg. & Med. Chem. (1997) 5:661-672, in Medicinal Research Reviews (2003) 23:32-47, in J. Org. Chem. (2000) 65:24-29 or in J. Am. Chem. Soc. (1996) 118:7653-7662.

Carbohydrate Research (2007) 342:558-566 discloses 5 cytidin-5′-yl[heteroaryl-phosphonatomethyl]-phosphates wherein heteroaryl may be thiazolyl, benzothiazolyl, benzoxazolyl, benzothienyl or thienyl, which are expected (but not demonstrated) to be sialyltransferase inhibitors. These compounds are obtained in a three-steps synthetic procedure starting from (a) a heteroaryl carbaldehyde, (b) diallyl H-phosphonate and (c) (N-acetyl-2′,3′-di-O-acetylcytidinyloxy)-cyanoethoxy-diisopropylaminophosphane. This document however does not teach any nucleoside or deoxynucleoside phosphothiolates or phosphoramidates. With regard to nucleoside phosphates, it does not disclose the presence of phosphonato groups being substituted with aryl, phosphonyl, sulfonyl or carboxyl.

Therefore, considering all aforementioned aspects of therapy directed to inhibit viral polymerases and reverse transcriptases, a nucleotide analogue that would not depend on activation by nucleoside/nucleotide kinases whilst serving as a natural substrate mimic, would be of a great interest.

### SUMMARY OF THE INVENTION

The present invention provides novel phosphate-modified nucleosides which can act as substrates of DNA- or RNA-polymerases and/or as antiviral agents.

The present invention provides novel phosphate-modified nucleosides that can be used as alternative (compared to natural NTPs or dNTPs) efficient substrates for DNA- or RNA-polymerases. In a particular embodiment, these phosphate-modified nucleotides are such that the pyrophosphate group of nucleosides/nucleotides is replaced by an easily leaving group, more particularly a leaving group in a nucleotidyl transfer mechanism. In a specific embodiment of the invention, this leaving group includes a carboxylic acid-, sulfate-, sulfonate-, phosphate- and/or phosphonate-containing group coupled to the nucleoside by a phosphoramide binding moiety. More particularly this leaving group contains two or more carboxylic acid, sulfate, sulfonate, phosphate or phosphonate groups which may be the same or different, and which are linked directly or via aliphatic chains containing 1, 2 or 3 carbon atoms to the nitrogen atom of the phosphoramide binding moiety.

### DETAILED DESCRIPTION OF THE INVENTION

According to a first broad aspect, the present invention encompasses phosphate-modified nucleosides represented by the structural formula (A):

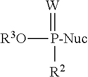

wherein

Nuc is a natural nucleoside or a nucleoside analogue, wherein said natural nucleoside or nucleoside analogue can be non-substituted or substituted as defined below;

R³ is selected from the group consisting of hydrogen, C₁₋₆ alkyl, C₃₋₆ cycloalkyl, aryl-C₁₋₆ alkyl, C₁₋₆ acyloxymethylene, C₁₋₆ alkoxycarbonyloxymethylene and 2-cyanoethyl, wherein said C₁₋₆ alkyl, C₃₋₆ cycloalkyl, C₁₋₆ acyloxymethylene, C₁₋₆ alkoxycarbonyl-oxymethylene or aryl-C₁₋₆ alkyl is optionally substituted with one or more, preferably 1, 2 or 3, substituents independently selected from the group consisting of halogen, OH, C₁₋₆ alkoxy, trifluoromethyl, trifluoromethoxy, nitro, cyano and amino;

W is O or S;

R² is represented by the structural formula (II):

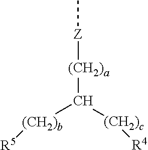

wherein

dotted lines represent the point of attachment of Z to the phosphorous atom P of the structural formula (A);

Z is selected from the group consisting of O, S, NH and NR₇; and

R⁷ is selected from the group consisting of C₁₋₆ alkyl, phenyl, benzyl and cyclohexyl;

a is 0 or 1;

b is 0, 1 or 2;

c is 0, 1 or 2 or 3;

R⁵ is selected from the group consisting of hydrogen; aryl; imidazolyl; P(O)(OH)₂; O—P(O)(OH)₂; S(O)₂(OH); O—S(O)₂(OH); and COOR⁶, wherein R⁶ is hydrogen or C₁₋₆ alkyl;

R⁴ is selected from the group consisting of P(O)(OH)₂, O—P(O)(OH)₂, S(O)₂(OH) and O—S(O)₂(OH); or, provided that Z is NH, a=b=0, c is 1 and R⁵ is COOH, R⁴ is selected from the group consisting of C₆H₅—OP(O)(OH)₂ wherein said C₆H₅ (phenyl) is substituted with fluoromethyl or difluoromethyl; C₆H₅—CHXP(O)(OH)₂; C₆H₅-QS(O)₂(CH═CH₂); C₆H₅-QV wherein said C₆H₅ (phenyl) is substituted with oxiran-2-yl or CH═CH₂; C₆H₅—C(═CH₂)V; CHXV and C(═CH₂)V;

X is chloro or bromo;

Q is a linking moiety selected from the group consisting of O, CH₂, (CH₂)₂ and CF₂;

V is selected from the group consisting of P(O)(OH)₂, S(O)₂(OH), SO₂NH₂, SO₂CH₃ and SO₂CF₃;

or R² is represented by the structural formula (V):

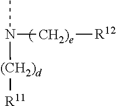

wherein
dotted lines represent the point of attachment of N to the phosphorous atom of formula (A);
d is 0, 1, 2, 3 or 4;
e is 0, 1, 2, or 3;
R¹¹ is selected from the group consisting of P(O)(OH)₂, O—P(O)(OH)₂, S(O)₂(OH) or O—S(O)₂(OH);
R¹² is selected from the group consisting of hydrogen; aryl; imidazolyl; P(O)(OH)₂; O—P(O)(OH)₂; S(O)₂(OH); O—S(O)₂(OH); and COOR⁶, wherein R⁶ is hydrogen or C₁₋₆ alkyl;
   and stereoisomers, pharmaceutically acceptable salts and pro-drugs thereof,
   provided that said phosphate-modified nucleoside is not one wherein R² is represented by the structural formula (V) and wherein d=0, e=O, R¹² is hydrogen or aryl, and R¹¹ is P(O)(OH)₂;
   provided that said phosphate-modified nucleoside is not one wherein R² is represented by the structural formula (II) wherein Z is O, a=0, b=0, R⁵ is aryl, c=0 and R⁴ is P(O)(OH)₂;
   provided that said phosphate-modified nucleoside is not one wherein R² is represented by the structural formula (II) wherein b and c are both 0, or wherein b and c are both 0 when Z is O; and
   provided that said phosphate-modified nucleoside is not one wherein R² is represented by the structural formula (V) wherein d is 0, or wherein d is 0 when Z is O.

According to a more specific embodiment of this first aspect of the invention, said natural nucleoside or nucleoside analogue (Nuc) is coupled via its 5′ position (referring to the standard numbering of atoms for cyclic sugar moieties) to the phosphorous atom P in the structural formula (A).

A second more specific aspect of the present invention relates to phosphate-modified nucleotides represented by the structural formula (I):

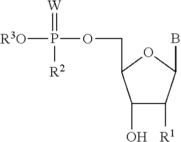

### Wherein

B is a pyrimidine or purine base, or an analogue thereof such as defined below), optionally substituted with one or two substituents independently selected from the group consisting of halogen, hydroxyl, sulfhydryl, methyl, ethyl, isopropyl, amino, methylamino, ethylamino, trifluoromethyl and cyano;
R¹ is H or OH;
R³ is selected from the group consisting of H, C₁₋₆ alkyl, C₃₋₆ cycloalkyl, aryl-C₁₋₆ alkyl, C₁₋₆ acyloxymethylene, C₁₋₆ alkoxycarbonyloxymethylene and 2-cyanoethyl, wherein said C₁₋₆ alkyl, C₃₋₆ cycloalkyl, C₁₋₆ acyloxymethylene, C₁₋₆ alkoxycarbonyloxymethylene or aryl-C₁₋₆ alkyl is optionally substituted with one or more, preferably 1, 2 or 3, substituents independently selected from the group consisting of halogen, OH, C₁₋₆ alkoxy, trifluoromethyl, trifluoromethoxy, nitro, cyano and amino;
W is O or S; and
R² is represented by the structural formula (II):

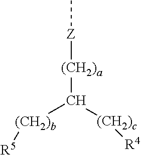

wherein

dotted lines represent the point of attachment of Z to the phosphorous atom P of the structural formula (I);

Z is selected from the group consisting of O; S; NH and NR';

R⁷ is selected from the group consisting of C₁₋₆ alkyl, phenyl, benzyl and cyclohexyl,

a is 0 or 1;

b is 0, 1 or 2;

c is 0, 1 or 2 or 3;

R⁵ is selected from the group consisting of hydrogen; aryl; imidazolyl; P(O)(OH)₂; O—P(O)(OH)₂; S(O)₂(OH); O—S(O)₂(OH); and COOR⁶, wherein R⁶ is hydrogen or C₁₋₆ alkyl;

R⁴ is selected from the group consisting of P(O)(OH)₂, O—P(O)(OH)₂, S(O)₂(OH) and O—S(O)₂(OH); or, provided that Z is NH, a=b=0, c is 1 and R⁵ is COOH, R⁴ is selected from the group consisting of C₆H₅—OP(O)(OH)₂ wherein said C₆H₅ (phenyl) is substituted with fluoromethyl or difluoromethyl; C₆H₅—CHXP(O)(OH)₂; C₆H₅-QS(O)₂(CH═CH₂); C₆H₅-QV wherein said C₆H₅ (phenyl) is substituted with oxiran-2-yl or CH═CH₂; C₆H₅—C(═CH₂)V; CHXV and C(═CH₂)V;

X is chloro or bromo;

Q is a linking moiety selected from the group consisting of O, CH₂, (CH₂)₂ and CF₂;

V is selected from the group consisting of P(O)(OH)₂, S(O)₂(OH), SO₂NH₂, SO₂CH₃ and SO₂CF₃;

or R² is represented by the structural formula (V):

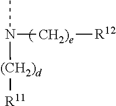

wherein
dotted lines represent the point of attachment of N to the phosphorous atom of formula (I);
d is 0, 1, 2, or 3;
e is 0, 1, 2, or 3;
R¹¹ is selected from the group consisting of P(O)(OH)₂, O—P(O)(OH)₂, S(O)₂(OH) and O—S(O)₂(OH);

R¹² is selected from the group consisting of aryl; imidazolyl; P(O)(OH)₂; O—P(O)(OH)₂; S(O)₂(OH); O—S(O)₂(OH); and COOR⁶, wherein R⁶ is hydrogen or C₁₋₆ alkyl;

and stereoisomers, pharmaceutically acceptable salts and pro-drugs thereof,
provided that said phosphate-modified nucleoside is not one wherein R² is represented by the structural formula (V) and wherein d=0, e=0, R¹² is hydrogen or aryl, and R¹¹ is P(O)(OH)₂;
provided that said phosphate-modified nucleoside is not one wherein R² is represented by the structural formula (II) wherein Z is O, a=0, b=0, R⁵ is aryl, c=0 and R⁴ is P(O)(OH)₂;
provided that said phosphate-modified nucleoside is not one wherein R² is represented by the structural formula (II) wherein b and c are both 0, or wherein b and c are both 0 when Z is O, and
provided that said phosphate-modified nucleoside is not one wherein R² is represented by the structural formula (V) wherein d is 0, or wherein d is 0 when Z is O.

In each of the structural formulae (A) and (I), W is preferably O (oxygen) but oxygen can be replaced by S (sulfur) by chemical reactions well known in the art.

According to another embodiment of the present invention, i.e. with respect to the structural formula (A) or the structural formula (I), the molecular weight of the group R₂ is not above 500.

In a particular embodiment, the present invention relates to phosphate-modified nucleosides represented by the structural formula (I) wherein R¹, R², R³ and W have any of the values as described herein, and wherein B is adenine; guanine; cytosine; thymine; uracil, or a substituted uracil as described below.

In another particular embodiment, the present invention relates to phosphate-modified nucleosides represented by the structural formula (A) wherein R², R³ and W have any of the values as described herein, and wherein Nuc is a natural nucleoside or nucleoside analogue wherein the nucleobase is adenine; guanine; cytosine; thymine; uracil, or a substituted uracil as described below.

In a particular embodiment, the present invention relates to phosphate-modified nucleosides represented by the structural formula (I) wherein B, R¹, R² and W have any of the values as described herein, and wherein R³ is H.

In another particular embodiment, the present invention relates to phosphate-modified nucleosides represented by the structural formula (A) wherein Nuc, R² and W have any of the values as described herein, and wherein R³ is H.

In a particular embodiment, the present invention relates to phosphate-modified nucleosides represented by the structural formula (I) wherein B, R¹, R² and R³ have any of the values as described herein, and wherein W is O.

In another particular embodiment, the present invention relates to phosphate-modified nucleosides represented by the structural formula (A) wherein Nuc, R² and R³ have any of the values as described herein, and wherein W is O.

In a particular embodiment, the present invention relates to phosphate-modified nucleosides represented by the structural formula (I) wherein B, R², R³ and W have any of the values as described herein, and wherein R¹ is H.

In another particular embodiment, the present invention also relates to the phosphate-modified nucleoside represented by the structural formula (I) wherein B, R², R³ and W have any of the values as described herein, and wherein R′ is OH.

In a particular embodiment, the present invention relates to phosphate-modified nucleosides represented by the structural formula (I) wherein B, R¹, R³ and W have any of the values described herein, wherein R² is represented by the structural formula (II) and wherein a is 0 or 1.

In another particular embodiment, the present invention also relates to the phosphate-modified nucleosides represented by the structural formula (A) wherein Nuc, R³ and W have any of the values described herein, wherein R² is represented by the structural formula (II) and wherein a is 0 or 1.

In a particular embodiment, the present invention relates to phosphate-modified nucleosides represented by the structural formula (I) wherein B, R¹, R³ and W have any of the values described herein, wherein R² is represented by the structural formula (II), and wherein c is 1.

In another particular embodiment, the present invention also relates to the phosphate-modified nucleosides represented by the structural formula (A) wherein Nuc, R³ and W have any of the values described herein, wherein R² is represented by the structural formula (II) and wherein c is 1.

In a particular embodiment, the present invention relates to phosphate-modified nucleosides represented by the structural formula (I) wherein B, R¹, R³ and W have any of the values described herein, wherein R² is represented by the structural formula (II) and wherein b is 0.

In another particular embodiment, the present invention also relates to the phosphate-modified nucleosides represented by the structural formula (A) wherein R³ and W have any of the values described herein, wherein R² is represented by the structural formula (II) and wherein b is 0.

In particular embodiments of the invention, the present invention relates to phosphate-modified nucleosides represented by the structural formula (I) wherein B, R¹, R², R³ and W have any of the values described herein, and wherein R⁵ or R¹² is COOH;

In other particular embodiments, the present invention relates to phosphate-modified nucleosides represented by the structural formula (A) wherein R², R³ and W have any of the values described herein, and wherein R⁵ or R¹² is COOH.

In other particular embodiments, the present invention relates to phosphate-modified nucleosides represented by the structural formula (A) wherein Nuc, R³ and W have any of the values described herein, wherein R² is represented by the structural formula (II), wherein R⁵ is COOH and wherein b is 0.

In another particular embodiment, the present invention relates to phosphate-modified nucleosides represented by the structural formula (I) wherein B, R¹, R³ and W have any of the values described herein, wherein R² is represented by the structural formula (II), wherein R⁵ is COOH and wherein b is 0.

In other particular embodiments, the present invention relates to phosphate-modified nucleosides represented by the structural formula (A) wherein R³ and W have any of the values described herein, wherein R² is represented by the structural formula (V), wherein R¹² is COOH and wherein e is 1.

In another particular embodiment, the present invention relates to phosphate-modified nucleosides represented by the structural formula (I) wherein B, R¹, R³ and W have any of the values described herein, wherein R² is represented by the structural formula (V), wherein R¹² is COOH and wherein e is 1.

In other particular embodiments, the present invention relates to phosphate-modified nucleosides represented by the structural formula (I) wherein B, R¹, R², R³ and W have any of the values described herein, and wherein R⁴ or R¹¹ is selected from the group consisting of P(O)(OH)₂, O—P(O)(OH)₂, S(O)₂(OH) and O—S(O)₂(OH).

In another particular embodiment, the present invention relates to phosphate-modified nucleosides represented by the structural formula (A) wherein Nuc, R², R³ and W have any of the values described herein, and wherein R⁴ or R¹¹ is selected from the group consisting of P(O)(OH)₂, O—P(O)(OH)₂, S(O)₂(OH) and O—S(O)₂(OH).

In other particular embodiments, the present invention relates to phosphate-modified nucleosides represented by the structural formula (I) wherein B, R¹, R³ and W have any of the values described herein, wherein R² is represented by the structural formula (V), wherein R¹¹ is P(O)(OH)₂, and wherein d is 1 or 2.

In other particular embodiments, the present invention relates to phosphate-modified nucleosides represented by the structural formula (I) wherein B, R¹, R³ and W have any of the values described herein, wherein R² is represented by the structural formula (II), wherein R⁴ is P(O)(OH)₂ or S(O)₂(OH), and wherein c is 1.

In other particular embodiments, the present invention relates to phosphate-modified nucleosides represented by the structural formula (A), or the structural formula (I), wherein B, Nuc, R², R³ and W have any of the values described herein, and wherein R⁴ or R¹¹ is selected from the group consisting of P(O)(OH)₂, O—P(O)(OH)₂, S(O)₂(OH) and O—S(O)₂(OH), and in more particular embodiments of the foregoing R⁴ or R¹¹ is P(O)(OH)₂ or S(O)₂(OH).

In other particular embodiments, the present invention relates to phosphate-modified nucleosides represented by the structural formula (A) wherein Nuc, R³ and W have any of the values described herein, wherein R² is represented by the structural formula (V), wherein R¹¹ is P(O)(OH)₂, and wherein d is 1 or 2.

In other particular embodiments, the present invention relates to phosphate-modified nucleosides represented by the structural formula (A) wherein Nuc, R³ and W have any of the values described herein, wherein R² is represented by the structural formula (II), wherein R⁴ is P(O)(OH)₂ or S(O)₂(OH), and wherein c is 1.

In a particular embodiment, the present invention relates to phosphate-modified nucleosides represented by the structural formula (I) wherein B, R¹, R³ and W have any of the values described herein, wherein R² is represented by the structural formula (V) and wherein d is 1 or 2 or 3 or 4.

in another particular embodiment, the present invention relates to phosphate-modified nucleosides represented by the structural formula (A) wherein Nuc, R³ and W have any of the values described herein, wherein R² is represented by the structural formula (V) and wherein d is 1 or 2 or 3 or 4.

In a particular embodiment, the present invention relates to phosphate-modified nucleosides represented by the structural formula (I) wherein B, R¹, R³ and W have any of the values described herein, wherein R² is represented by the structural formula (V) and wherein e is 0 or 1.

In another particular embodiment, the present invention relates to phosphate-modified nucleosides represented by the structural formula (A) wherein Nuc, R³ and W have any of the values described herein, wherein R² is represented by the structural formula (V) and wherein e is 0 or 1.

In a particular embodiment, the present invention relates to phosphate-modified nucleosides represented by the structural formula (I) wherein R¹, R², R³ and W have any of the values described herein, and wherein B is a pyrimidine or purine base analogue as described in the definitions section below, in particular 5-azapyrimidine, 5-azacytosine, 7-deazapurine, 7-deazaadenine, 7-deazaguanine, or 7-deaza-8-azapurines.

In another particular embodiment, the present invention relates to phosphate-modified nucleosides represented by the structural formula (A) wherein R², R³ and W have any of the values as described herein, and wherein Nuc is a natural nucleoside or nucleoside analogue wherein the base is a pyrimidine or purine base analogue as described in the definitions section below, in particular 5-azapyrimidine, 5-azacytosine, 7-deazapurine, 7-deazaadenine, 7-deazaguanine, or 7-deaza-8-azapurines.

In a particular embodiment of the foregoing, the present invention relates to phosphate-modified nucleoside represented by the structural formula (I) wherein B, R¹, R³ and W have any of the values as described herein, and wherein R² is a phosphonate containing group coupled by a phosphoramide binding, and in a more particular embodiment of the foregoing said R² additionally contains a carboxylic acid containing group, and in an even more particular embodiment of the foregoing these phosphonate and carboxylic acid containing groups are linked directly or via alkyl groups containing 1, 2 or 3 C (carbon) atoms to the N of the phosphoramide binding, and in an even more particular embodiment of the foregoing R² is NH—CH(COOH)(CH₂—P(O)(OH)₂).

In another particular embodiment of the invention, the present invention relates to phosphate-modified nucleoside represented by the structural formula (A) wherein R³ and W have any of the values as described herein, and wherein R² is a phosphonate containing group coupled by a phosphoramide binding, and in a more particular embodiment of the foregoing said R² additionally contains a carboxylic acid containing group, and in an even more particular embodiment of the foregoing these phosphonate and carboxylic acid containing groups are linked directly or via alkyl groups containing 1, 2 or 3 (carbon atoms to the nitrogen atom of the phosphoramide binding moiety, and in an even more particular embodiment of the foregoing R² is NH—CH(COOH)(CH₂—P(O)(OH)₂).

In another particular embodiment of the invention, with reference to the structural formula (II), Z is preferably O; NH or NR⁷ wherein R⁷ is methyl, ethyl, propyl or butyl.

In particular embodiments of the invention the aryl group R⁵ or R¹² is a C₆ aryl (phenyl) optionally substituted with one or more substituents, preferably 1, 2 or 3 substituents, independently selected from the group consisting of halogen, amino, trifluoromethyl, hydroxyl, sulfhydryl, nitro, (C₁-C₆)alkoxy, trifluoromethoxy, cyano and (CH₂)_{q}—COOR⁶, wherein R⁶ is hydrogen or C₁₋₆ alkyl, and q is 0, 1 or 2; in a more particular embodiment of the foregoing said C₆ aryl (phenyl) is substituted with 1, 2 or 3 groups (CH₂)_{q}—COOR⁶, wherein R⁶ is hydrogen or C₁₋₆ alkyl, and q is 0, 1 or 2.

In another particular embodiment of the invention, R³ is H and the aryl group R⁵ or R¹² is a C₆ aryl (phenyl) substituted with 1, 2 or 3 groups (CH₂)_{q}—COOR⁶, wherein R⁶ is hydrogen or C₁₋₆ alkyl, and q is 0, 1 or 2.

In a yet more particular embodiment of the invention, R³ is H and the aryl group R⁵ or R¹² is a C₆ aryl (phenyl) group substituted with two carboxylic acid groups.

In a yet more particular embodiment of the invention, R³ is H and R² is represented by the structural formula (II), wherein Z is O, S, NH or NCH₃, and R⁵ is 2-carboxyphenyl or 3-carboxyphenyl (derived from phthalic acid or isophthalic acid).

In another particular embodiment of the invention, R³ is H and R² is represented by the structural formula (V), wherein R¹² is 2-carboxyphenyl or 3-carboxyphenyl (derived from phthalic acid or isophthalic acid).

In another particular embodiment of the invention, R³ is H and R² is represented by the structural formula (II), wherein a is 0, b is 0, c is 1 and R⁵ is COOH and R⁴ is P(O)(OH)₂ or S(O)₂(OH).

In a particular embodiment of the invention, R² is represented by the structural formula (II), wherein R⁵ is COOCH₃ or COOCH₂CH₃.

In another particular embodiment of the invention, R³ is H and R² is represented by the structural formula (II), wherein a is 0, b is 0, c is 1 and R⁵ is COOCH₃ or COOCH₂CH₃ and R⁴ is P(O)(OH)₂ or S(O)₂(OH), and in a more particular embodiment of the foregoing R⁴ is P(O)(OH)₂.

In a particular embodiment of the foregoing, the present invention relates to phosphate-modified nucleoside represented by the structural formula (A) or the structural formula (I) wherein Nuc (in the structural formula A) is a natural nucleoside or nucleoside analogue wherein the base (noted as B in the structural formula I) is a pyrimidine analogue represented by the structural formula (C):

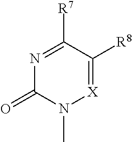

wherein
R⁷ is selected from the group consisting of OH, SH, NH₂, NHCH₃ and NHC₂H₅;
R⁸ is selected from the group consisting of hydrogen, methyl, ethyl, isopropyl, amino, ethylamino, trifluoromethyl, cyano and halogen; and
X is CH or N; and

in yet another particular embodiment of the foregoing, the present invention also relates to the phosphate-modified nucleoside represented by the structural formula (A) or the structural formula (I) wherein Nuc (in the structural formula A) is a natural nucleoside or nucleoside analogue wherein the base (noted as B in the structural formula I) is a purine analogue represented by the structural formula (D):

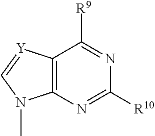

wherein
R⁹ is selected from the group consisting of H, OH, SH, NH₂, and NHCH₃;
R¹⁰ is selected from the group consisting of hydrogen, methyl, ethyl, hydroxyl, amino and halogen; and
Y is CH or N.

In a particular embodiment of the present invention, the novel phosphate-modified nucleoside is 2′-deoxyadenosine-5′-(3-phosphono-L-alanine)phosphoramidate (3-phosphono-L-Ala-dAMP); 2′-deoxyguanosine-5′-(3-phosphono-L-alanine) phosphoramidate (3-phosphono-L-Ala-dGMP); 2′-deoxythymidine-5′-(3-phosphono-L-alanine)phosphoramidate (3-phosphono-L-Ala-dTMP); 2′-deoxyuridine-5′-(3-phosphono-L-alanine)phosphoramidate (3-phosphono-L-Ala-d UM P); 2′-deoxycytidine-5′-(3-phosphono-L-alanine)phosphoramidate (3-phosphono-L-Ala-dCMP); adenosine-5′-(3-phosphono-L-alanine)phosphoramidate (3-phosphono-L-Ala-AMP); guanosine-5′-(3-phosphono-L-alanine)phosphoramidate (3-phosphono-L-Ala-GMP); 5-methyluridine-5′-(3-phosphono-L-alanine)phosphoramidate (3-phosphono-L-Ala-m5 uMP); uridine-5′-(3-phosphono-L-alanine)phosphoramidate (3-phosphono-L-Ala-UMP); or cytidine-5′-(3-phosphono-L-alanine)phosphoramidate (3-phosphono-L-Ala-CMP).

Another aspect of the present invention relates to the use of the phosphate-modified nucleosides represented by the structural formulae (A) and (I), including any one of the above-referred specific embodiments thereof, as a substrate for DNA- or RNA-polymerases, these polymerases being either wild-type (naturally occurring) or mutated according to common knowledge in the art. In a particular embodiment, said DNA- or RNA-polymerases are selected from Therminator DNA polymerase, KF (exo⁻) DNA polymerase, or Reverse Transcriptase (e.g. HIV-RT) or mutated forms of these enzymes. If needed, the enzymes as described herein above can be mutated, using common knowledge in the art, in order to better adapt to the novel phosphate-modified nucleoside disclosed in this invention. In a particular embodiment, the present invention relates to the use of the phosphate-modified nucleosides of the invention, as a substrate for DNA- or RNA-polymerases in bacteriae or in vitro. In another particular embodiment, said DNA- or RNA-polymerase originates from a micro-organism or from bacterial or viral origin.

In a particular embodiment, the phosphate-modified nucleosides of this invention being represented by the structural formulae (A) and (I), including any one of the above-referred specific embodiments thereof, can be used to build in at least 1, 2 or 3 nucleotides in a growing DNA- or RNA-strand.

In another particular embodiment, the phosphate-modified nucleosides of this invention being represented by the structural formulae (A) and (I), including any one of the above-referred specific embodiments thereof, can be used to build in at most 1, 2 or 3 nucleotides in a growing DNA- or RNA-strand.

In another particular embodiment, the phosphate-modified nucleosides of this invention being represented by the structural formulae (A) and (I), including any one of the above-referred specific embodiments thereof, can be used to build in at most 300 nucleotides in a growing DNA- or RNA-strand.

In yet another particular embodiment, the phosphate-modified nucleosides of this invention being represented by the structural formulae (A) and (I), including any one of the above-referred specific embodiments thereof, can be used with a mixture of natural dNTPs or NTPs (ATP,CTP,GTP,UTP,TTP) as a substrate for DNA/RNA-polymerases, more in particular to build in 1-300 (e.g. 2-300) nucleotides in a growing DNA- or RNA-strand.

The present invention also relates to the use of the phosphate-modified nucleoside represented by the structural formulae (A) and (I), including any one of the above-referred specific embodiments thereof, for the enzymatic production of oligonucleotides, peptides or proteins.

In a particular embodiment, the phosphate-modified nucleosides of the invention being represented by the structural formulae (A) and (I), including any one of the above-referred specific embodiments thereof, can be used for in vitro production of DNA or RNA. In another particular embodiment, the phosphate-modified nucleosides of the invention being represented by the structural formulae (A) and (I), including any one of the above-referred specific embodiments thereof, can also be used for in vitro production of peptides or proteins. In another particular embodiment the phosphate-modified nucleosides of the invention being represented by the structural formulae (A) and (I), including any one of the above-referred specific embodiments thereof, can be used for PCR (polymerase chain reaction).

In yet another particular embodiment, the phosphate-modified nucleosides of the invention being represented by the structural formulae (A) and (I), including any one of the above-referred specific embodiments thereof, can be used as a substrate for the growth of wild type and/or mutated bacteriae. In a particular embodiment, the phosphate-modified nucleotides of the invention being represented by the structural formulae (A) and (I), including any one of the above-referred specific embodiments thereof, can be used as a substrate for the growth of bacteriae with mutated DNA/RNA polymerase, preferably wherein the mutation is suitable to better adapt better to the new substrate.

In view of their antiviral activity discussed below, another aspect of the present invention relates to a pharmaceutical, veterinary or non-pharmaceutical composition comprising an anti-virally effective amount of a phosphate-modified nucleoside of the invention being represented by the structural formulae (A) and (I), including any one of the above-referred specific embodiments thereof. In a particular embodiment, said pharmaceutical, veterinary or non-pharmaceutical composition may further comprise an aqueous solution and optionally one or more buffering agents. In a particular embodiment, said pharmaceutical, veterinary or non-pharmaceutical composition may further comprise one or more natural NTPs or dNTPs (e.g. ATP, CTP, GTP, UTP or TTP).

Another aspect of the invention relates to the use of the non-pharmaceutical composition of the invention as a substrate to build in at least 1, 2 or 3 nucleotides in a growing DNA- or RNA-strand.

Yet another aspect of the invention relates to the use of the phosphate-modified nucleosides of the invention, being represented by the structural formulae (A) and (I), including any one of the above-referred specific embodiments thereof, in a non-human living organism for sustaining growth, survival or proliferation of said organism. In a particular embodiment said organism is selected from the group consisting of a virus, a bacterium, an archaeon and an eukaryote, and in a more particular embodiment said eukaryote is selected from the group consisting of yeast, mold, fungus, microalga, multicellular plant and protist.

Yet another aspect of the invention relates to a method for the production of oligonucleotides, RNA, DNA, peptides and/or proteins by using the phosphate-modified nucleotides of the invention, being represented by the structural formulae (A) and (I), including any one of the above-referred specific embodiments thereof.

Another aspect of the present invention relates to compounds represented by the structural formulae (A) and (I), including any one of the above-referred specific embodiments thereof, having antiviral activity, specifically to these compounds that inhibit the replication of viruses (such as, but not limited to, viruses belonging to the order Herpesvirales, in particular the family Herpesviridae, the family Alloherpesviridae or the family Malacoherpesviridae), in particular retroviruses, and even more specifically to these compounds that inhibit the replication of HIV-1 or HIV-2.

Another aspect of the present invention relates to the compounds represented by the structural formulae (A) and (I), including any one of the above-referred specific embodiments thereof, for use as a medicine, more particularly for use to treat or prevent a viral infection in a mammal, even more particularly to treat or prevent HIV infection in a mammal such as a human being.

Another aspect of the invention relates to pharmaceutical compositions comprising an anti-virally effective amount of at least one compound being represented by the structural formulae (A) and (I), including any one of the above-referred specific embodiments thereof, in combination with one or more pharmaceutically acceptable excipients being well known in the art for the formulation of phosphate nucleosides. The invention further relates to the use of the compounds represented by the structural formulae (A) and (I), including any one of the above-referred specific embodiments thereof, in the manufacture of a medicament useful for the treatment of viral infections (e.g. from a virus belonging to the order Herpesvirales), more specifically for the treatment of a retroviral infection such as a HIV-1 or HIV-2 infection.

The present invention also relates to a method of treatment or prevention of a viral infection in a mammal, comprising the administration of a therapeutically effective (e.g. anti-virally effective or replication-inhibiting) amount of a compound of this invention represented by the structural formulae (A) and (I), including any one of the above-referred specific embodiments thereof, optionally in combination with one or more pharmaceutically acceptable excipients. In a more particular embodiment of the foregoing, said viral infection is a HIV infection. In a more particular embodiment of the foregoing, said mammal is a human being.

Still another aspect of the invention relates to processes for the preparation of the phosphate-modified nucleosides according to the first or the second aspect of the present invention. Such processes will be explained with reference to the synthetic schemes 1 to 7 hereinafter. In one embodiment of said aspect, one method comprises the step of coupling a nucleotide monophosphate (NMP) such as shown on the top left part of schemes 2 and 6 or at the left part of schemes 4-5 with an amine reactant comprising the easily leaving group of this invention preferably in a protected form such as an acid ester to produce a phosphoramidate nucleoside analogue, as depicted for instance in schemes 2, 4 and 5 below. Deprotection of the ester group of this nucleoside analogue by means of a deprotecting agent such as, but not limited to, an alkali hydroxide, e.g. potassium hydroxide or sodium hydroxide such as 0.4 M NaOH, provides the desired phosphoramidate nucleoside of this invention in its acidic form. An alternative process for the preparation of the phosphate-modified nucleosides of the invention comprises the synthetic step as shown in scheme 3 below or in the following scheme 1:

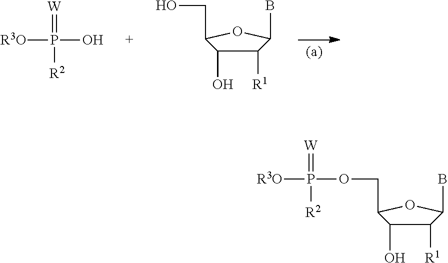

wherein (a) schematically represents the presence in the reaction mixture of an effective amount of a suitable catalyst for the condensation of the 5′-OH and phosphate acid groups, such as but not limited to an arylsulfonylhalide, e.g. an optionally substituted phenylsulfonylchloride. Phosphates, phosphorothioates and phosphoramidates (as shown on the left part of scheme 1) wherein R² and R³ are as defined in the structural formulae (A) and (I), including any one of the above-referred specific embodiments thereof, to be used as starting meterials in this first method are well known in the art or may be produced according to one or more of the synthetic methods as described by Scheit in Nucleotide Analogs, J. Wiley, New-York (1980) or by Vaghefi in Nucleoside Triphosphate and their analogs, Taylor & Francis, CRC Press (2005), the contents of which are incorporated herein by reference.

The following scheme 2 for the preparation of phosphate-modified nucleosides represented by the structural formula (I) wherein R² is represented by the structural formula (II) involves three reaction steps, illustrative reagents and conditions thereof being as follows:

step (a): presence of a carbodiimide coupling agent such as EDAC, H;O, room temperature, 2 to 5 hours, under argon atmosphere;

step (b) 1.4M K₂CO₃ in a MeOH/H₂O (1:1 volume ratio) solvent mixture; room temperature, 2 hours, under argon atmosphere;

step (c) 0.4M NaOH in a MeOH/H₂O (1:1 volume ratio) solvent mixture, room temperature, 15 hours, under argon atmosphere.

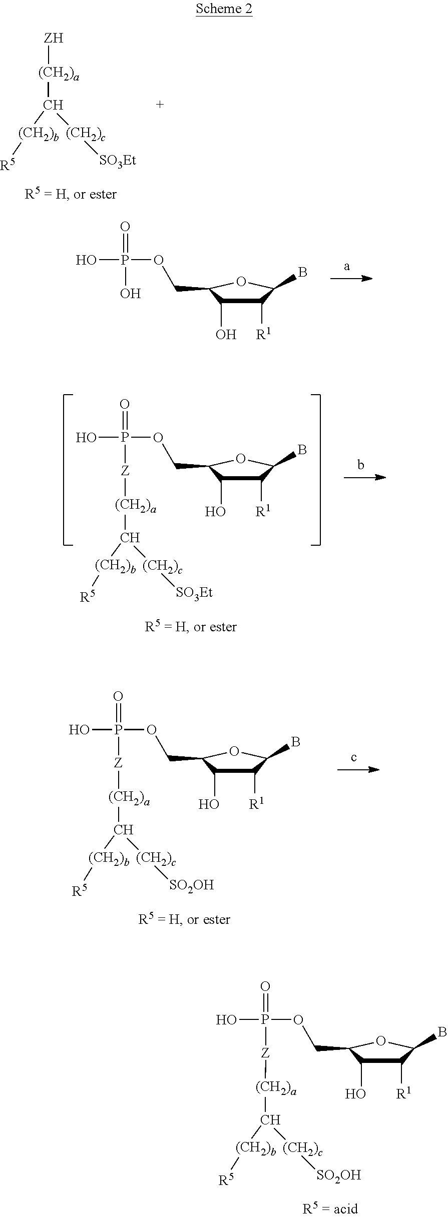

The following scheme 3 for the preparation of phosphate-modified nucleosides represented by the structural formula (I) wherein R² is represented by the structural formula (II) involves a single reaction step (d), illustrative reagents and conditions thereof being as follows: N-ethylmorpholine, H₂O, room temperature, 3 days, under argon atmosphere.

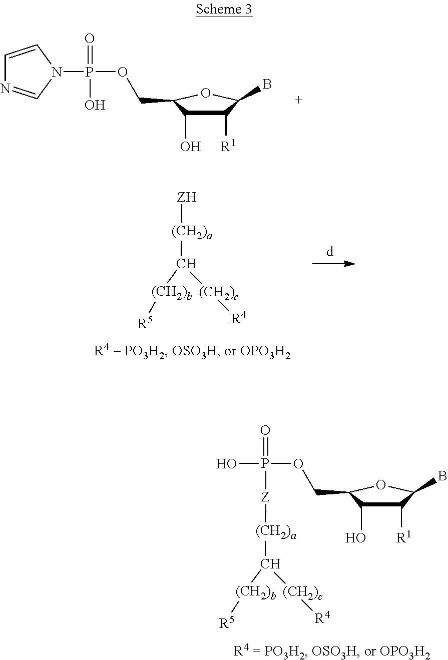

Scheme 4 below depicts a synthetic route for preparing phosphate-modified nucleosides represented by the structural formula (I) wherein R² is represented by the structural formula (V):

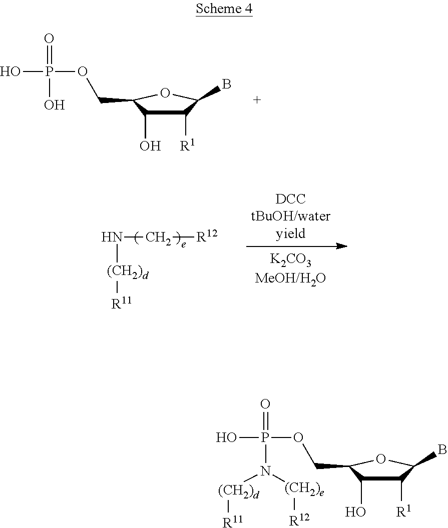

### BRIEF DESCRIPTION OF THE DRAWINGS

FIG. 1 shows a representation of the chemical structures of five phosphoramidates analogues of this invention: Tau-dAMP (1), L-Cys-dAMP (2), 3-phosphono-L-Ala-dAMP (3), aphospho-L-Ser-dAMP (4) and O-sulfonato-L-Ser-dAMP (5).

FIG. 2 shows the incorporation of the illustrative compounds of this invention, Tau-dAMP (1) (FIG. 2A), L-Cys-dAMP (2) (FIG. 2B), and 3-phosphono-L-Ala-dAMP (3) (FIGS. 2C and 2D) into P₁T₁ (125 nM) by HIV-1 RT with compound concentrations and time intervals (minutes) as indicated, [HIV-1 RT]=0.025 U/μL; dATP (10 μM) incorporation is shown at right for reference.

FIG. 3 shows the elongation of the primer/template P₁T₂ (125 nM) by HIV-1 RT at different phosphoramidate concentrations and time intervals (minutes) as indicated, [HIV-1 RT]=0.025 U/μl, with L-Cys-dAMP (2) and 3-phosphono-L-Ala-dAMP (3), or no nucleotide substrate (blank), dATP (50 μM) being shown at right for reference.

FIG. 4 shows the incorporation of 3-phosphono-L-Ala-dAMP (3) into P₁T₁ (125 nM) by Taq DNA polymerase at different concentrations and time intervals (minutes) as indicated, [Taq]=0.025 U/μL; d ATP (10 μM) incorporation is shown at right for reference.

FIG. 5 shows the incorporation of three further illustrative compounds of this invention (compounds 6, 7 and 8 in FIGS. 5A, 5B and 5C respectively) into P₁T₁ (125 nM) by HIV-1 RT with compound concentrations and time intervals (minutes) as indicated, [HIV-1 RT]=0.025 U/μL; dATP (10 μM) incorporation is shown at right for reference.

FIG. 6 shows activation of AZT monophosphate by a novel leaving group moiety of this invention (top) and an application of a phosphoramidate moiety for in vivo delivery and activation of AZT monophosphate (bottom).

### DEFINITIONS

The term “nucleoside” conventionally refers to natural glycosylamines consisting of a nucleobase bound to a ribose or deoxyribose sugar via a β-glycosidic linkage such as cytidine, urisine, adenosine, guanosine and thymidine. The term “nucleoside analogue” as used herein refers to known nucleosides consisting of a cyclic sugar moiety (such as defined below) linked to nucleobase or analogue thereof such as a pyrimidine or purine nucleobase as defined below, including modifications wherein the sugar ring moety is modified or substituted and/or wherein the nucleobase is modified or substituted as defined below. Nucleoside analogues wherein the natural sugar moiety is modified include but are not limited to:

nucleoside analogues wherein the ribose sugar is replaced with another monocyclic sugar such as, but not limited to, arabinofuranose, arabinopyranose, xylofuranose, xylopyranose, lyxofuranose, lyxopyranose, α-D-threofuranose; threose nucleic acids (TNA) also referred as α-threofuranosyl nucleosides are described for example by Orgel in Science 290 (5495) 1306-1307 and by Schong et al in Science 290 (5495) 1347-1351, the contents of which are incorporated herein by reference;

nucleoside analogues wherein the ribose sugar is replaced with a bicyclic or tricyclic sugar, such as locked nucleic acids (LNA) wherein the ribose moiety is modified with at least an extra bridge connecting the 2′-oxygen and 4′-carbon atoms, wherein the bridge locks the ribose in the 3′-endo conformation; LNA are described for example in WO 99/14226, the content of which is incorporated herein by reference;

nucleoside analogues wherein the (deoxy)ribose sugar is unsaturated (dehydrodeoxyribose) or substituted with one or more conventional substituents (e.g. azido) for nucleoside technology such as, but not limited to, 2′,3′-deoxy-3′-azidoribose.

The term “sugar” as used herein includes ribose and deoxyribose, linked by the oxygen atom at position 5 of the ribose or deoxyribose moiety to the phosphorous atom P in the structural formula (A) or the structural formula (I), but is not limited thereto and also includes modifications and variants of the ribose or deoxyribose moiety. Such modifications are well known to those skilled in the art, examples being pentofuranoses such as listed above, unsaturated and/or substituted monocyclic sugars such as, but not limited to, 2,3-deoxy-3-azido-ribose, and also bicyclic or tricyclic sugars such as present in locked nucleic acids (LNA).

The term “pyrimidine or purine base” as used herein is exemplary of nucleobases which may be present in the nucleoside analogues of this invention and includes, but is not limited to, adenine, thymine, cytosine, uracyl, guanine and 2,6-diaminopurine and analogues and derivatives thereof. A purine or pyrimidine base as used herein includes a purine or pyrimidine base found in naturally occurring nucleosides as mentioned above. An analogue thereof is a base which mimics such naturally occurring bases in such a way that their structures (the kinds of atoms and their arrangement) are similar to the naturally occurring bases but may either possess additional or lack certain of the functional properties of the naturally occurring bases. Such analogues include those derived by replacement of a CH moiety by a nitrogen atom (e.g. 5-azapyrimidines such as 5-azacytosine) or vice versa (e.g., 7-deazapurines, such as 7-deazaadenine or 7-deazaguanine) or both (e.g., 7-deaza, 8-azapurines). By derivatives of such bases or analogues are meant those bases wherein ring substituents are either incorporated, removed, or modified by conventional substituents known in the art, e.g. halogen, hydroxyl, amino, C₁₋₆ alkyl and other non reactive and biocompatible substituents. Such purine or pyrimidine bases, and analogues and derivatives thereof, are well known to those skilled in the art, e.g. as shown at pages 20-38 of WO 03/093290, Horlacher et al. in PNAS (1995) 92:6329; and U.S. Pat. No. 6,617,106, more specifically on pages 3-5), the contents of which are incorporated herein by reference.

In particular purine and pyrimidine analogues B for the purpose of the present invention may be selected from the group comprising pyrimidine bases represented by the structural formula (C):

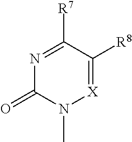

wherein

R⁷ is selected from the group consisting of OH, SH, NH₂, NHCH₃ and NHC₂H₅;

R⁸ is selected from the group consisting of hydrogen, methyl, ethyl, isopropyl, amino, ethylamino, trifluoromethyl, cyano and halogen; and

X is CH or N;

and purine bases and analogues represented by the structural formula (D):

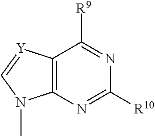

wherein:
R⁹ is selected from the group consisting of hydrogen, OH, SH, NH₂, and NH-Me;
R¹⁰ is selected from the group consisting of hydrogen, methyl, ethyl, hydroxyl, amino and halogen; and
Y is CH or N.

Just as a few non-limiting examples of pyrimidine analogues, can be named substituted uracils with the formula (C) wherein X is CH, R⁷ is hydroxyl, and R⁸ is methyl, ethyl, isopropyl, amino, ethylamino, trifluoromethyl, cyano, fluoro, chloro, bromo and iodo.

Non-limiting examples of nucleobase analogues for use in this invention include adenosine, cytidine, guanosine, uridine, 2′-deoxyadenosine, 2′-deoxycytidine, 2′-deoxyguanosine, thymidine, inosine, 9-(3-D-arabinofuranosyl) adenine, 1-(D-arabinofuranosyl) cytosine, 9-(D-arabinofuranosyl) guanine, 1-(D-arabinofuranosyl) uracil, 9-(D-arabinofuranosyl) hypoxanthine, 1-(D-arabinofuranosyl) thymine, 3′-azido-3′-deoxythymidine, 3′-azido-2′,3′-dideoxyuridine, 3′-azido-2′,3′-dideoxycytidine, 3′-azido-2′,3′-dideoxyadenosine, 3′-azido-2′,3′-dideoxyguanosine, 3′-azido-2′,3′-dideoxyinosine, 3′-deoxythymidine, 2′,3′-dideoxyuridine, 2′,3′-dideoxyinosine, 2′,3′-dideoxyadenosine, 2′,3′-dideoxycytidine, 2′,3′-dideoxyguanosine, 9-(2,3-dideoxy-1-(3-D-ribofuranosyl)-2,6-diaminopurine, 3′-deoxy-2′,3′-didehydrothymidine, 2′,3′-didehydro-2′,3′-dideoxyuridine, 2′,3′-didehydro-2′,3′-dideoxycytidine, 2′,3′-didehydro-2′,3′-dideoxyadenosine, 2′,3′-didehydro-2′,3′-dideoxyguanosine, 2′,3′-didehydro-2′,3′-dideoxyinosine, 3-deazaadenosine, 3-deazaguanosine, 3-deazainosine, 7-deazaadenosine, 7-deazaguanosine, 7-deazainosine, 6-azauridine, 6-azathymidine, 6-azacytidine, 5-azacytidine, 9-(D-ribofuranosyl)-6-thiopurine, 6-methylthio-9-(D-ribofuranosyl) purine, 2-amino-9-(D-ribofuranosyl)-6-thiopurine, 2-amino-6-methylthio-9-(D-ribofuranosyl) purine, 5-fluorocytidine, 5-iodocytidine, 5-bromocytidine, 5-chlorocytidine, 5-fluorouridine, 5-iodouridine, 5-bromouridine, 5-chlorouridine, 2′-C-methyladenosine, 2′-C-methylcytidine, 2′-C-methylguanosine, 2′-C-methylinosine, 2′-C-methyluridine, 2′-C-methylthymidine, 2′-deoxy-2′-fluoroadenosine, 2′-deoxy-2′-fluorocytidine, 2′-deoxy-2′-fluoroguanosine, 2′-deoxy-2′-fluorouridine, 2′-deoxy-2′-fluoroinosine, 2′-a-fluorothymidine, 2′-deoxy-2′-fluoroarabinoadenosine, 2′-deoxy-2′-fluoroarabinocytidine, 2′-deoxy-2′-fluoroarabinoguanosine, 2′-deoxy-2′-fluoroarabinouridine, 2′-deoxy-2′-fluoroarabinoinosine, 2′-fluorothynnidine, 2′-O-methyladenosine, 2′-O-methylcytidine, 2′-O-methylguanosine, 2′-O-methylinosine, 2′-O-5-dimethyluridine, L-uridine, L-inosine, 2′,3′-didehydro-2′,3′-dideoxy-L-cytidine, 2′,3′-didehydro-3′-dideoxy-L-thymidine, 2′,3′-didehydro-2′,3′-dideoxy-L-adenosine, 2′,3′-didehydro-2′,3′-dideoxy-L-guanosine, 2′,3′-didehydro-2′,3′-dideoxy-L-5-fluorocytidine, 2′-deoxy-2′,2′-difluorocytidine, 9-(D-arabinofuranosyl)-2-fluoroadenine, 2′-deoxy-2′(Z)-fluoromethylenecytidine, 2′,3′-dideoxy-3′-thiacytidine, 1-D-ribofuranosyl-1,2,4-triazole-3-carboxamide, 1-L-ribofuranosyl-1,2,4-triazole-3-carboxamide, 1-D-ribofuranosyl-1,3-imidazolium-5-olate, 1-L-ribofuranosyl-1,3-imidazolium-5-olate, 1-D-ribofuranosyl-5-ethynylimidazole-4-carboxamide, 1-L-ribofuranosyl-5-ethynylimidazole-4-carboxamide, 1-(2-deoxy-2-fluoro-D-arabinofuranosyl)-5-iodouracil, 1-(2-deoxy-2-fluoro-D-arabinofuranosyl)-5-iodocytosine, 1-(2-deoxy-2-fluoro-L-arabinofuranosyl)-5-methyluracil, 1-D-arabinofuranosyl-5-(2-bromovinyl) uracil, 5-(2-bromovinyl)-2′-deoxyuridine,5-trifluoromethylthymidine, 1-D-arabinofuranosyl-5-propynyluracil, 1-(2-deoxy-2-fluoro-1-D-arabinofuranosyl)-5-ethyluracil, 2′,3′-dideoxy-3′-fluoroguanosine, 3′-deoxy-3′-fluorothymidine, 9-[2,3-bis(hydroxymethyl)-1-cyclobutyl] adenine, 9-[2,3-bis(hydroxymethyl)-1-cyclobutyl] guanine, 9-[2,3-bis(hydroxymethyl)-1-cyclobutyl] guanine, 9-[2,3-bis(hydroxymethyl)-1-cyclobutyl] adenine, (1R,3S,4R)-9-(3-hydroxy-4-hydroxymethylcyclopent-1-yl) guanine, (1S,2R,4R)-9-(1-hydroxy-2-hydroxymethylcyclopent-4-yl) guanine, (2R,4R)-9-(2-hydroxymethyl-1,3-dioxolan-4-yl)-2,6-diaminopurine, (2R,4R)-1-(2-hydroxymethyl-1,3-dioxolan-4-yl) cytosine, (2R,4R)-9-(2-hydroxymethyl-1,3-dioxolan-4-yl) guanine, (2R,4R)-1-(2-hydroxymethyl-1,3-dioxolan-4-yl)-5-fluorocytosine, (1R,2S,4S)-9-(4-hydroxy-3-hydroxymethyl-2-methylenecyclopent-4-yl] guanine, and (1S,3R,4S)-9-(3-hydroxy-4-hydroxymethyl-5-methylenecylopent-1-yl] guanine.

The term “C₁₋₆alkyl” as used herein refers to normal, secondary, or tertiary hydrocarbon chains having from 1 to 6 carbon atoms. Examples thereof are methyl, ethyl, 1-propyl, 2-propyl, 1-butyl, 2-methyl-1-propyl(1-Bu), 2-butyl (s-Bu) 2-methyl-2-propyl (t-Bu), 1-pentyl (n-pentyl), 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, n-pentyl, n-hexyl.

As used herein and unless otherwise stated, the term “cycloalkyl” means a monocyclic saturated hydrocarbon monovalent group having from 3 to 10 carbon atoms, such as for instance cyclopropyl, cyclobutyl, cyclopentyl, cyclohexyl, cycloheptyl, cyclooctyl, or a C₇₋₁₀ polycyclic saturated hydrocarbon monovalent group having from 7 to 10 carbon atoms such as, for instance, norbornyl, fenchyl, trimethyltricycloheptyl or adamantyl.

The term “C₁₋₆ alkoxy” as used herein refers to substituents wherein a carbon atom of a C₁₋₆ alkyl group (such as defined herein), is attached to an oxygen atom through a single bond such as, but not limited to, methoxy, ethoxy, propoxy, isopropoxy, butoxy, iso-butoxy, sec-butoxy, tert-butoxy, pentoxy, 3-pentoxy, or n-hexyloxy.

As used herein, and unless otherwise stated, the term “aryl” designates any mono- or polycyclic aromatic monovalent hydrocarbon groupl having from 6 up to 30 carbon atoms such as, but not limited to phenyl, naphthyl, anthracenyl, phenantracyl, fluoranthenyl, chrysenyl, pyrenyl, biphenylyl, terphenyl, picenyl, indenyl, biphenyl, indacenyl, benzocyclobutenyl, benzocyclooctenyl and the like, including benzo-fused cycloalkyl radicals (the latter being as defined above) such as, for instance, indanyl, tetrahydronaphthyl, fluorenyl and the like, all of the said groups being optionally substituted with one or more substituents independently selected from the group consisting of halogen, amino, trifluoromethyl, hydroxyl, sulfhydryl, nitro, C₁₋₆ alkoxy, trifluoromethoxy, cyano and (CH₂)_{q}—COOR⁶, wherein R⁶ is selected from the group consisting of hydrogen, C₁₋₆ alkyl and benzyl, and q is selected from 0, 1, and 2, such as for instance carboxyphenyl, phthalic acid (1,2-dicarboxyphenyl), isophthalic acid (1,3-dicarboxyphenyl), 4-fluorophenyl, 4-chlorophenyl, 3,4-dichlorophenyl, 4-cyanophenyl, 2,6-dichlorophenyl, 2-fluorophenyl, 3-chlorophenyl, 3,5-dichlorophenyl and the like.

As used herein with respect to a substituting group, and unless otherwise stated, the term “aryl-C₁₋₆ alkyl” refers to an aliphatic saturated hydrocarbon monovalent group (preferably a C₁₋₆ alkyl group such as defined above) onto which an aryl group (such as defined above) is linked via a carbon atom, and wherein the said aliphatic group and/or the said aryl group may be optionally substituted with one or more substituents independently selected from the group consisting of halogen, amino, hydroxyl, sulfhydryl, C₁₋₆ alkyl, C₁₋₆ alkoxy, trifluoromethyl, trifluoromethoxy, nitro and carboxylic acid, such as but not limited to benzyl, 4-chlorobenzyl, 4-fluorobenzyl, 2-fluorobenzyl, 3,4-dichlorobenzyl, 2,6-dichlorobenzyl, 3-methylbenzyl, 4-methylbenzyl, 4-tert-butylbenzyl, phenylpropyl, 1-naphthylmethyl, phenylethyl and the like.

As used herein and unless otherwise stated, the term halogen means any atom selected from the group consisting of fluorine, chlorine, bromine and iodine.

Any substituent designation that is found in more than one place in a modified nucleoside of this invention may be independently selected at each place.

The term “amino acid” as used herein refers to any “natural amino acid” (Alanine (ala), Arginine (Arg), Asparagine (asn), Aspartic acid (Asp), Cysteine (cys), Glutamine (gin), Glutamic acid (glu), Glycine (gly), Histidine (his), Hydroxylysine (Hyl), Hydroxyproline (Hyp), Isoleucine (ile), Leucine (leu), Lysine (lys), Methionine (met), Phenylalanine (phe), Proline (pro), Serine (ser), Threonine (thr), Tryptophan (trp), Tyrosine (tyr), Valine (val)) in D or L conformation (but, within the context of this invention, preferably the L conformation), as well as to “non-natural (or synthetic) amino acids” (e.g., but not limited to, phosphoserine, phosphothreonine, phosphotyrosin, hydroxyproline, gamma-carboxyglutamate; hippuric acid, octahydroindole-2-carboxylic acid, statine, 1,2,3,4,-tetrahydroisoquinoline-3-carboxylic acid, penicillamine, ornithine, citruline, α-methyl-alanine, para-benzoylphenylalanine, phenylglycine, propargylglycine, sarcosine, tert-butylglycine, and amino-acids bearing sulfonic and/or phosphonic groups such as specifically described below). This term also comprises natural and non-natural amino acids being protected at their carboxylic terminus (e.g. as a C₁₋₆ alkyl, phenyl or benzyl ester or as an amide, such as for example, a mono-C₁₋₆alkyl or di-(C₁₋₆ alkyl) amide. Other suitable carboxy protecting groups are known to those skilled in the art (see for example, T. W. Greene, Protecting Groups In Organic Synthesis; Wiley: New York, (1981) and references cited therein, the content of which is incorporated herein by reference).

It will be appreciated by those skilled in the art that certain modified nucleosides of this invention having a chiral center may exist in, and be isolated in, optically active and racemic forms. Some modified nucleosides may exhibit polymorphism. It is to be understood that the present invention encompasses any racemic, optically-active, polymorphic, or stereoisomeric form, or mixtures thereof in any proportions, of a modified nucleoside of this invention, which may possess the useful properties described herein. It is well known in the art how to prepare optically active forms (for example, by resolution of the racemic form by recrystallization techniques, by synthesis from optically-active starting materials, by chiral synthesis, or by chromatographic separation using a chiral stationary phase).

As used herein and unless otherwise stated, the term “stereoisomer” refers to all possible different isomeric as well as conformational forms which the compounds of formula (I) and formula (A) may possess, in particular all possible stereochemically and conformationally isomeric forms, all diastereomers, enantiomers and/or conformers of the basic molecular structure. Some compounds of the present invention may exist in different tautomeric forms, all of the latter being included within the scope of the present invention.

As used herein and unless otherwise stated, the term “enantiomer” means each individual optically active form of a compound of the invention, having an optical purity or enantiomeric excess (as determined by methods standard in the art) of at least 80% (i.e. at least 80% of one enantiomer and at most 20% of the other enantiomer), preferably at least 90% and more preferably at least 98%.

Certain of the phosphate-modified nucleotides herein when substituted with appropriate selected functionalities are capable of acting as pro-drugs. These are labile functional groups which separate from an active inhibitory phosphate-modified nucleotide during metabolism, systemically, inside a cell, by hydrolysis, enzymatic cleavage, or by some other process (Bundgaard “Design and Application of Pro-drugs” in Textbook of Drug Design and Development (1991), Eds. Harwood Academic Publishers, pp. 113-191, the content of which is incorporated herein by reference). These pro-drug moieties can serve to enhance solubility, absorption and lipophilicity to optimize drug delivery, bioavailability and efficacy. A “pro-drug” is thus a covalently modified analogue of a therapeutically-active modified nucleoside of this invention. A pro-drug moiety can also be therapeutically active in its own right.

Exemplary suitable pro-drug moieties include, but are not limited to, esters of nucleosides like the POM (pivaloyloxymethyl), POC (isopropyloxycarbonyloxymethyl) and SATE (S-acyl-2-thioethyl) esters.

The term “salt” as used herein, refers to the ionic product of a reaction between an acid and a base. Salts of the compounds having structural formula I may be formed at any acid or base functionality within the compound, in particular, R³, R⁴ and R⁵ may represent or comprise an acid or base functionality. In particular, salts of the compounds represented by the structural formula (I) or the structural formula (A) may be formed as follows. When R³ is hydrogen, it is acidic and may therefore engage in salt formation with an inorganic or organic base. R⁴ and R⁵ comprise acid functionalities such as carboxylic groups (i.e. —COOH), which can equally engage in salt formation with an organic or inorganic base. Alternatively, R⁴ and R⁵ may comprise base functionalities such as the imidazolyl, which in turn can engage in salt formation with an organic or inorganic acid. The term “pro-drug”, as used herein, relates to an inactive or active derivative of a compound represented by the structural formula (I) or the structural formula (A) as defined herein above or any one of their specific embodiments, which undergoes spontaneous or enzymatic transformation within the body of an animal, e.g. a mammal such as a human being, in order to release the pharmacologically active form of the compound. For a comprehensive review, reference is made to Rautio J. et al. (“Pro-drugs: design and clinical applications” in Nature Reviews Drug Discovery (2008) doi: 10.1038/nrd2468, the content of which is incorporated herein by reference). In particular for the purpose of the present invention, pro-drugs of the compounds represented by the structural formula (A) or the structural formula (I), including any one of the above-described specific embodiments thereof, may be formed as follows. When R³ is H, a free phosphate acid function is available for pro-drug formation as described in detail by Hecker et al. (“Prodrugs of phosphates and phosphonates” Journal of Medicinal Chemistry (2008) doi: 10.1021/jm701260b, the content of which is incorporated herein by reference). R⁴ and R⁵ comprise acid functionalities such as carboxylic acid groups (i.e. —COOH) which may be used for the formation of a pro-drug. Such carboxylic acid pro-drug may occur in the form of an ester, in particular acyloxyalkyl esters (e.g. pivaloyloxymethyl ester (POM)) or S-acylthioethyl (SATE) esters, a carbonate, a carbamate or an amide, such as amino acid pro-drugs.

The term “peptide” as used herein refers to a sequence of 2 to 50 amino acids (e.g. as defined hereinabove) or peptidyl residues. The sequence may be linear or cyclic. Preferably a peptide comprises 2 to 25, or 5 to 20 amino acids.

The term “oligonucleotide” as used herein refers to a polynucleotide formed by a plurality of linked nucleotide units. The nucleotide units each include a nucleoside unit linked together via a phosphate linking group. These nucleotides can be natural or modified in their phosphate, sugar or nucleobase group. The oligonucleotide may be naturally occurring or non naturally occurring.

The term “polymerase” as used herein refers to an enzyme that can synthesize DNA or RNA from a DNA or RNA template and includes but is not limited to Therminator DNA polymerase, KF(exo⁻)DNA polymerase and HIV Reverse Transcriptase.

### Biological Applications of the Invention

Novel phosphoramidates, phospho-esters and phospho-thioesters according to this invention may be used as alternative substrates and biotechnology tools.

Fast emerging applications of modified nucleosides as biotechnology tools also require new and efficient ways to synthesize DNA and RNA building blocks such as nucleoside triphosphates and amidites for the use, for example, in PCR, labeling, or enzymatic incorporation of nucleotides, and in the automated DNA synthesis, respectively. Furthermore, some biotechnology applications require incorporation of a nucleotide by enzymatic means using DNA or RNA polymerases. However, at times, due to chemical nature and modifications present in the modified nucleosides, triphosphate synthesis is not always feasible and/or provides insufficient and low yields. Therefore, carboxyl-containing and/or phosph(on)ate- or sulf(on)ate-containing groups coupled to a nucleoside monophosphate through a phosphoramidate (P—N) bond can serve as an alternative or substitute group to a pyrophosphate moiety. However, fitting into an active site and the subsequent nucleotidyl transfer may be less efficient for such carboxyl-containing and/or phosph(on)ate- or sulf(on)ate-containing phosphoramidate (e.g. 3-phosphono-L-Ala-dAMP) compared to the natural substrates/dNTPs (e.g. dATP) for the natural polymerase/enzyme. In this situation, mutated polymerases can be used to increase the efficiency of recognition and incorporation of the compounds of this invention. Such an embodiment of the invention with mutated polymerases can be used to specifically select or grow bacteriae by using these carboxyl- and/or phosph(on)ate- or sulf(on)ate-containing phosphoramidate nucleosides as a substrate. An additional advantage of this application is that polymerases that demonstrated efficient recognition and incorporation of carboxyl-containing phosphoramidate nucleosides in our studies are also shown to tolerate various sugar modifications and unnatural nucleobases quite well. Therefore, the enzymatic synthesis of DNA and, RNA sequences containing unnatural nucleobases can be accomplished whilst avoiding at times cumbersome nucleoside triphosphate synthesis and purification.

The phosphoramidates, phospho-esters and phospho-thioesters of this invention are also useful as antiviral compounds.

The compounds of the invention can be efficiently used for the treatment of viral infections, particularly retroviral infections, more particularly Human Immunodeficiency Virus (HIV) infections, in particular of Human Immunodeficiency Virus type 1 (HIV-1). When using one or more phosphate-modified nucleosides represented by the structural formula (I) or the structural formula (A) as defined herein, including any one of the specific embodiments thereof:

the active ingredients of the compound(s) may be administered to the mammal (including a human being) to be treated by any means well known in the art, i.e. orally, intranasally, subcutaneously, intramuscularly, intradermally, intravenously, intra-arterially, parenterally or by catheterization;

the therapeutically effective amount of the preparation of the compound(s), especially for the treatment of viral infections in humans and other mammals, preferably is a HIV enzyme inhibiting amount. More preferably, it is a HIV replication inhibiting amount or a HIV enzyme (in particular reverse transcriptase) inhibiting amount of the phosphate-modified nucleosides represented by the structural formula (I) or the structural formula (A) as defined herein, including any one of the specific embodiments thereof, corresponding to an amount which ensures a plasma level of between 1 μg/ml and 100 mg/ml, optionally of 10 mg/ml. Depending upon the pathologic condition to be treated and the patient's condition, the said effective amount may be divided into several sub-units per day or may be administered at more than one day intervals.

The present invention further relates to a method for preventing or treating a viral infection, e.g. a retroviral infection, in a subject or patient by administering to the patient in need thereof a therapeutically effective amount, e.g. an anti-virally effective amount, of a compounds of the present invention. The therapeutically effective amount of the compound(s), especially for the treatment of viral infections in humans and other mammals, preferably is a HIV enzyme inhibiting amount. More preferably, it is a HIV replication inhibiting amount or a HIV enzyme (in particular reverse transcriptase) inhibiting amount of the derivative(s) of represented by the structural formula (I) or the structural formula (A) as defined herein, including any one of the specific embodiments thereof. Depending upon the pathologic condition to be treated and the patient's condition, the said effective amount may be divided into several sub-units per day or may be administered at more than one-day intervals.

The present invention also relates to a combination of different antiviral drugs of the invention or to a combination of the antiviral drugs of the invention with other drugs that exhibit anti-HIV.

The invention also relates to a combined preparation of antiviral drugs which may be either:

A) a composition comprising
(a) a combination of two or more of the compounds of the present invention, including any one of the specific embodiments thereof, and
(b) optionally one or more pharmaceutical excipients or pharmaceutically acceptable carriers,
   for simultaneous, separate or sequential use in the treatment or prevention of a viral infection, e.g. a retroviral infection, or
   B) a composition comprising
(c) one or more anti-viral agents, and
(d) at least one compound of the present invention, including any one of the specific embodiments thereof, and
(e) optionally one or more pharmaceutical excipients or pharmaceutically acceptable carriers,
   for simultaneous, separate or sequential use in the treatment or prevention of a viral infection, e.g. a retroviral infection.

Suitable anti-viral agents (c) for inclusion into the antiviral combined preparations of this invention include for instance, inhibitors of BVDV or HCV replication respectively, such as interferon-alpha (pegylated or not), ribavirin and other selective inhibitors of the replication of HCV, such as a compound disclosed in EP-1,162,196, WO 03/010141, WO 03/007945, WO 03/010140 or WO 00/204425 (the contents of which are incorporated herein by reference) and/or an inhibitor of flaviviral protease and/or one or more additional flavivirus polymerase inhibitors.

The pharmaceutical composition or combined preparation with activity against viral infection according to this invention may contain a compound of the present invention, including any one of the specific embodiments thereof, over a broad content range depending on the contemplated use and the expected effect of the preparation. Generally, the content of the compound of the present invention (including any one of the specific embodiments thereof) in the combined preparation is within the range of 0.1 to 99.9% by weight, preferably from 1 to 99% by weight, more preferably from 5 to 95% by weight.

When using a pharmaceutical composition or combined preparation:

the active ingredients may be administered to the mammal (including a human) to be treated by any means well known in the art, i.e. orally, intranasally, subcutaneously, intramuscularly, intradermally, intravenously, intra-arterially, parenterally or by catheterization; and/or

the therapeutically effective amount of each of the active agents, especially for the treatment of viral infections in humans and other mammals, particularly is a HIV enzyme inhibiting amount.

When applying a combined preparation, the active ingredients may be administered simultaneously but it is also beneficial to administer them separately or sequentially, for instance within a relatively short period of time (e.g. within about 24 hours) in order to achieve their functional fusion in the body to be treated.

The invention also relates to the compounds of the formulae described herein, including any one of the above-described specific embodiments thereof, for use in the inhibition of the proliferation of other viruses than HIV-1, particularly for the inhibition of other members of the family of the retroviruses.

The present invention further provides veterinary compositions comprising at least one active ingredient as above defined together with a veterinary carrier therefor. Veterinary carriers are materials useful for the purpose of administering the composition and may be solid, liquid or gaseous materials which are otherwise inert or acceptable in the veterinary art and are compatible with the active ingredient. These veterinary compositions may be administered orally, parenterally or by any other desired route.

More generally, the invention relates to the compounds represented by the structural formula (I) or the structural formula (A), including any one of the above-described specific embodiments thereof, being useful as agents having biological activity (particularly antiviral activity) or as diagnostic agents.

The compounds of the present invention may for instance be bound covalently to an insoluble matrix and used for affinity chromatography separations, depending on the nature of the groups of the compounds, for example compounds with pendant aryl are useful in hydrophobic affinity separations.

Those of skill in the art will also recognize that the compounds of the invention may exist in many different protonation states, depending on, among other things, the pH of their environment. While the structural formulae provided herein depict the compounds in only one of several possible protonation states, it will be understood that these structures are illustrative only, and that the invention is not limited to any particular protonation state—any and all protonated forms of the compounds are intended to fall within the scope of the invention.

The term “pharmaceutically acceptable salts” as used herein means the therapeutically active non-toxic salt forms which the compounds represented by the structural formula (I) or the structural formula (A), including any one of the above-described specific embodiments thereof, are able to form. Therefore, the compounds of this invention optionally comprise salts of the compounds herein, especially pharmaceutically acceptable non-toxic salts containing, for example, Na⁺, Li⁺, K⁺, Ca²⁺ and Mg²⁺. Such salts may include those derived by combination of appropriate cations such as alkali and alkaline earth metal ions or ammonium and quaternary amino ions with an acid anion moiety, typically a carboxylic acid. The compounds of the invention may bear multiple positive or negative charges. The net charge of the compounds of the invention may be either positive or negative. Any associated counter ions are typically dictated by the synthesis and/or isolation methods by which the compounds are obtained. Typical counter ions include, but are not limited to ammonium, sodium, potassium, lithium, halides, acetate, trifluoroacetate, etc., and mixtures thereof. It will be understood that the identity of any associated counter ion is not a critical feature of the invention, and that the invention encompasses the compounds in association with any type of counter ion. Moreover, as the compounds can exist in a variety of different forms, the invention is intended to encompass not only forms of the compounds that are in association with counter ions (e.g., dry salts), but also forms that are not in association with counter ions (e.g., aqueous or organic solutions). Metal salts typically are prepared by reacting the metal hydroxide with a compound of this invention. Examples of metal salts which are prepared in this way are salts containing Li⁺, Na⁺, and K⁺. A less soluble metal salt can be precipitated from the solution of a more soluble salt by addition of the suitable metal compound. In addition, salts may be formed from acid addition of certain organic and inorganic acids to basic centers, typically amines, or to acidic groups. Examples of such appropriate acids include, for instance, inorganic acids such as hydrohalic acids, e.g. hydrochloric or hydrobromic acid, sulfuric acid, nitric acid, phosphoric acid and the like; or organic acids such as, for example, acetic, propanoic, hydroxyacetic, 2-hydroxypropanoic, 2-oxopropanoic, lactic, pyruvic, oxalic (i.e. ethanedioic), malonic, succinic (i.e. butanedioic acid), maleic, fumaric, malic, tartaric, citric, methanesulfonic, ethanesulfonic, benzenesulfonic, p-toluenesulfonic, cyclohexanesulfamic, salicylic (i.e. 2-hydroxybenzoic), p-aminosalicylic and the like. Furthermore, this term also includes the solvates which the compounds represented by the structural formula (I) or the structural formula (A) including any one of the specific embodiments thereof, as well as their salts are able to form, such as for example hydrates, alcoholates and the like. Finally, it is to be understood that the compositions herein comprise compounds of the invention in their unon-ionized, as well as zwitterionic form, and combinations with stoichiometric amounts of water as in hydrates.

Also included within the scope of this invention are the salts of the parental compounds with one or more amino acids, especially the naturally-occurring amino acids found as protein components. The amino acid typically is one bearing a side chain with a basic or acidic group, e.g., lysine, arginine or glutamic acid, or a neutral group such as glycine, serine, threonine, alanine, isoleucine, or leucine.

The compounds of the invention also include physiologically acceptable salts thereof. Examples of physiologically acceptable salts of the compounds of the invention include salts derived from an appropriate base, such as an alkali metal (for example, sodium), an alkaline earth (for example, magnesium), ammonium and NA₄⁺ (wherein A is C₁-C₄ alkyl). Physiologically acceptable salts of an hydrogen atom or an amino group include salts of organic carboxylic acids such as acetic, benzoic, lactic, fumaric, tartaric, maleic, malonic, malic, isethionic, lactobionic and succinic acids; organic sulfonic acids, such as methanesulfonic, ethanesulfonic, benzenesulfonic and p-toluenesulfonic acids; and inorganic acids, such as hydrochloric, sulfuric, phosphoric and sulfamic acids. Physiologically acceptable salts of a compound containing a hydroxy group include the anion of said compound in combination with a suitable cation such as Na⁺ and NA₄⁺ (wherein A typically is independently selected from H or a C₁-C₄ alkyl group). However, salts of acids or bases which are not physiologically acceptable may also find use, for example, in the preparation or purification of a physiologically acceptable compound. All salts, whether or not derived form a physiologically acceptable acid or base, are within the scope of the present invention.

As used herein and unless otherwise stated, the term “enantiomer” means each individual optically active form of a compound of the invention, having an optical purity or enantiomeric excess (as determined by methods standard in the art) of at least 80% (i.e. at least 80% of one enantiomer and at most 20% of the other enantiomer), preferably at least 90% and more preferably at least 98%.

The term “isomers” as used herein means all possible isomeric forms, including tautomeric and sterochemical forms, which the compounds represented by the structural formula (A) or the structural formula (I) may possess, but not including position isomers. Typically, the structures shown herein exemplify only one tautomeric or resonance form of the compounds, but the corresponding alternative configurations are contemplated as well. Unless otherwise stated, the chemical designation of compounds denotes the mixture of all possible stereochemically isomeric forms, said mixtures containing all diastereomers and enantiomers (since the compounds of formula A and formula I may have at least one chiral center) of the basic molecular structure, as well as the stereochemically pure or enriched compounds. More particularly, stereogenic centers may have either the R- or S-configuration, and multiple bonds may have either cis- or trans-configuration. Pure isomeric forms of the said compounds are defined as isomers substantially free of other enantiomeric or diastereomeric forms of the same basic molecular structure. In particular, the term “stereoisomerically pure” or “chirally pure” relates to compounds having a stereoisomeric excess of at least about 80% (i.e. at least 80% of one isomer and at most 20% of the other possible isomers), preferably at least 90%, more preferably at least 94% and most preferably at least 97%. The terms “enantiomerically pure” and “diastereomerically pure” should be understood in a similar way, having regard to the enantiomeric excess, respectively the diastereomeric excess, of the mixture in question. Separation of stereoisomers is accomplished by standard methods known to those in the art. One enantiomer of a compound of the invention can be separated substantially free of its opposing enantiomer by a method such as formation of diastereomers using optically active resolving agents (“Stereochemistry of Carbon Compounds,” (1962) by E. L. Eliel, McGraw Hill; Lochmuller, C. H., (1975) J. Chromatogr., 113:(3) 283-302). Separation of isomers in a mixture can be accomplished by any suitable method, including: (1) formation of ionic, diastereomeric salts with chiral compounds and separation by fractional crystallization or other methods, (2) formation of diastereomeric compounds with chiral derivatizing reagents, separation of the diastereomers, and conversion to the pure enantiomers, or (3) enantiomers can be separated directly under chiral conditions. Under method (1), diastereomeric salts can be formed by reaction of enantiomerically pure chiral bases such as brucine, quinine, ephedrine, strychnine, a-methyl-b-phenylethylamine (amphetamine), and the like with asymmetric compounds bearing acidic functionality, such as carboxylic acid and sulfonic acid. The diastereomeric salts may be induced to separate by fractional crystallization or ionic chromatography. For separation of the optical isomers of amino compounds, addition of chiral carboxylic or sulfonic acids, such as camphorsulfonic acid, tartaric acid, mandelic acid, or lactic acid can result in formation of the diastereomeric salts. Alternatively, by method (2), the substrate to be resolved may be reacted with one enantiomer of a chiral compound to form a diastereomeric pair (Eliel, E. and Wilen, S. (1994) Stereochemistry of Organic Compounds, John Wiley & Sons, Inc., p. 322). Diastereomeric compounds can be formed by reacting asymmetric compounds with enantiomerically pure chiral derivatizing reagents, such as menthyl derivatives, followed by separation of the diastereomers and hydrolysis to yield the free, enantiomerically enriched xanthene. A method of determining optical purity involves making chiral esters, such as a menthyl ester or Mosher ester, a-methoxy-a-(trifluoromethyl)phenyl acetate (Jacob III. (1982) J. Org. Chem. 47:4165), of the racemic mixture, and analyzing the NMR spectrum for the presence of the two atropisomeric diastereomers. Stable diastereomers can be separated and isolated by normal- and reverse-phase chromatography following methods for separation of atropisomeric naphthyl-isoquinolines (Hoye, T., WO96/15111). Under method (3), a racemic mixture of two asymmetric enantiomers is separated by chromatography using a chiral stationary phase. Suitable chiral stationary phases are, for example, polysaccharides, in particular cellulose or amylose derivatives. Commercially available polysaccharide based chiral stationary phases are ChiralCel™ CA, OA, 0B5, OC5, OD, OF, OG, OJ and OK, and Chiralpak™ AD, AS, OP(+) and OT(+). Appropriate eluents or mobile phases for use in combination with said polysaccharide chiral stationary phases are hexane and the like, modified with an alcohol such as ethanol, isopropanol and the like.

The terms cis and trans are used herein in accordance with Chemical Abstracts nomenclature and include reference to the position of the substituents on a ring moiety. The absolute stereochemical configuration of the compounds of formula A and formula I may easily be determined by those skilled in the art while using well-known methods such as, for example, X-ray diffraction.

The compounds of the invention may be formulated with conventional carriers and excipients, which will be selected in accord with ordinary practice. Tablets will contain excipients, glidants, fillers, binders and the like. Aqueous formulations are prepared in sterile form, and when intended for delivery by other than oral administration generally will be isotonic. Formulations optionally contain excipients such as those set forth in the “Handbook of Pharmaceutical Excipients” (1986) and include ascorbic acid and other antioxidants, chelating agents such as EDTA, carbohydrates such as dextrin, hydroxyalkylcellulose, hydroxyalkylmethylcellulose, stearic acid and the like.

Subsequently, the term “pharmaceutically acceptable carrier” as used herein means any material or substance with which the active ingredient is formulated in order to facilitate its application or dissemination to the locus to be treated, for instance by dissolving, dispersing or diffusing the said composition, and/or to facilitate its storage, transport or handling without impairing its effectiveness. The pharmaceutically acceptable carrier may be a solid or a liquid or a gas which has been compressed to form a liquid, i.e. the compositions of this invention can suitably be used as concentrates, emulsions, solutions, granulates, dusts, sprays, aerosols, suspensions, ointments, creams, tablets, pellets or powders.

Suitable pharmaceutical carriers for use in the said pharmaceutical compositions and their formulation are well known to those skilled in the art, and there is no particular restriction to their selection within the present invention. They may also include additives such as wetting agents, dispersing agents, stickers, adhesives, emulsifying agents, solvents, coatings, antibacterial and antifungal agents (for example phenol, sorbic acid, chlorobutanol), isotonic agents (such as sugars or sodium chloride) and the like, provided the same are consistent with pharmaceutical practice, i.e. carriers and additives which do not create permanent damage to mammals. The pharmaceutical compositions of the present invention may be prepared in any known manner, for instance by homogeneously mixing, coating and/or grinding the active ingredients, in a one-step or multi-steps procedure, with the selected carrier material and, where appropriate, the other additives such as surface-active agents. They may also be prepared by micronisation, for instance in view to obtain them in the form of microspheres usually having a diameter of about 1 to 10 μm, namely for the manufacture of microcapsules for controlled or sustained release of the active ingredients.

Suitable surface-active agents, also known as emulgent or emulsifier, to be used in the pharmaceutical compositions of the present invention are non-ionic, cationic and/or anionic materials having good emulsifying, dispersing and/or wetting properties. Suitable anionic surfactants include both water-soluble soaps and water-soluble synthetic surface-active agents. Suitable soaps are alkaline or alkaline-earth metal salts, unsubstituted or substituted ammonium salts of higher fatty acids (C₁₀-C₂₂), e.g. the sodium or potassium salts of oleic or stearic acid, or of natural fatty acid mixtures obtainable form coconut oil or tallow oil. Synthetic surfactants include sodium or calcium salts of polyacrylic acids; fatty sulphonates and sulphates; sulphonated benzimidazole derivatives and alkylarylsulphonates. Fatty sulphonates or sulphates are usually in the form of alkaline or alkaline-earth metal salts, unsubstituted ammonium salts or ammonium salts substituted with an alkyl or acyl radical having from 8 to 22 carbon atoms, e.g. the sodium or calcium salt of lignosulphonic acid or dodecylsulphonic acid or a mixture of fatty alcohol sulphates obtained from natural fatty acids, alkaline or alkaline-earth metal salts of sulphuric or sulphonic acid esters (such as sodium lauryl sulphate) and sulphonic acids of fatty alcohol/ethylene oxide adducts. Suitable sulphonated benzimidazole derivatives preferably contain 8 to 22 carbon atoms. Examples of alkylarylsulphonates are the sodium, calcium or alcanolamine salts of dodecylbenzene sulphonic acid or dibutyl-naphtalenesulphonic acid or a naphtalene-sulphonic acid/formaldehyde condensation product. Also suitable are the corresponding phosphates, e.g. salts of phosphoric acid ester and an adduct of p-nonylphenol with ethylene and/or propylene oxide, or phospholipids. Suitable phospholipids for this purpose are the natural (originating from animal or plant cells) or synthetic phospholipids of the cephalin or lecithin type such as e.g. phosphatidylethanolamine, phosphatidylserine, phosphatidylglycerine, lysolecithin, cardiolipin, dioctanyl-phosphatidylcholine, dipalmitoylphosphatidylcholine and their mixtures.

Suitable non-ionic surfactants include polyethoxylated and polypropoxylated derivatives of alkylphenols, fatty alcohols, fatty acids, aliphatic amines or amides containing at least 12 carbon atoms in the molecule, alkylarenesulphonates and dialkylsulphosuccinates, such as polyglycol ether derivatives of aliphatic and cycloaliphatic alcohols, saturated and unsaturated fatty acids and alkylphenols, said derivatives preferably containing 3 to 10 glycol ether groups and 8 to 20 carbon atoms in the (aliphatic) hydrocarbon moiety and 6 to 18 carbon atoms in the alkyl moiety of the alkylphenol. Further suitable non-ionic surfactants are water-soluble adducts of polyethylene oxide with polypropylene glycol, ethylenediamino-polypropylene glycol containing 1 to 10 carbon atoms in the alkyl chain, which adducts contain 20 to 250 ethyleneglycol ether groups and/or 10 to 100 propyleneglycol ether groups. Such compounds usually contain from 1 to 5 ethyleneglycol units per propyleneglycol unit. Representative examples of non-ionic surfactants are nonylphenol-polyethoxyethanol, castor oil polyglycolic ethers, polypropylene/polyethylene oxide adducts, tributylphenoxypolyethoxyethanol, polyethyleneglycol and octylphenoxypolyethoxy-ethanol. Fatty acid esters of polyethylene sorbitan (such as polyoxyethylene sorbitan trioleate), glycerol, sorbitan, sucrose and pentaerythritol are also suitable non-ionic surfactants.

Suitable cationic surfactants include quaternary ammonium salts, particularly halides, having 4 hydrocarbon radicals optionally substituted with halo, phenyl, substituted phenyl or hydroxy; for instance quaternary ammonium salts containing as N-substituent at least one C₈-C₂₂ alkyl radical (e.g. cetyl, lauryl, palmityl, myristyl, oleyl and the like) and, as further substituents, unsubstituted or halogenated lower alkyl, benzyl and/or hydroxy-lower alkyl radicals.

A more detailed description of surface-active agents suitable for this purpose may be found for instance in “McCutcheon's Detergents and Emulsifiers Annual” (MC Publishing Crop., Ridgewood, New Jersey, 1981), “Tensid-Taschenbucw', 2^{nd} ed. (Hanser Verlag, Vienna, 1981) and “Encyclopaedia of Surfactants” (Chemical Publishing Co., New York, 1981).

Compounds of the invention and their physiologically acceptable salts (hereafter collectively referred to as the active ingredients) may be administered by any route appropriate to the condition to be treated, suitable routes including oral, rectal, nasal, topical (including ocular, buccal and sublingual), vaginal and parenteral (including subcutaneous, intramuscular, intravenous, intradermal, intrathecal and epidural). The preferred route of administration may vary with for example the condition of the recipient. While it is possible for the active ingredients to be administered alone it is preferable to present them as pharmaceutical formulations. The formulations, both for veterinary and for human use, of the present invention comprise at least one active ingredient, as above described, together with one or more pharmaceutically acceptable carriers therefore and optionally other therapeutic ingredients. The carrier(s) optimally are “acceptable” in the sense of being compatible with the other ingredients of the formulation and not deleterious to the recipient thereof. The formulations include those suitable for oral, rectal, nasal, topical (including buccal and sublingual), vaginal or parenteral (including subcutaneous, intramuscular, intravenous, intradermal, intrathecal and epidural) administration. The formulations may conveniently be presented in unit dosage form and may be prepared by any of the methods well known in the art of pharmacy. Such methods include the step of bringing into association the active ingredient with the carrier which constitutes one or more accessory ingredients. In general the formulations are prepared by uniformly and intimately bringing into association the active ingredient with liquid carriers or finely divided solid carriers or both, and then, if necessary, shaping the product.

Formulations of the present invention suitable for oral administration may be presented as discrete units such as capsules, cachets or tablets each containing a predetermined amount of the active ingredient; as a powder or granules; as solution or a suspension in an aqueous liquid or a non-aqueous liquid; or as an oil-in-water liquid emulsion or a water-in-oil liquid emulsion. The active ingredient may also be presented as a bolus, electuary or paste.

A tablet may be made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, preservative, surface active or dispersing agent. Molded tablets may be made by molding in a suitable machine a mixture of the powdered compound moistened with an inert liquid diluent. The tablets may optionally be coated or scored and may be formulated so as to provide slow or controlled release of the active ingredient therein. For infections of the eye or other external tissues e.g. mouth and skin, the formulations are optionally applied as a topical ointment or cream containing the active ingredient(s) in an amount of, for example, 0.075 to 20% w/w (including active ingredient(s) in a range between 0.1% and 20% in increments of 0.1% w/w such as 0.6% w/w, 0.7% w/w, etc), preferably 0.2 to 15% w/w and most preferably 0.5 to 10% w/w. When formulated in an ointment, the active ingredients may be employed with either a paraffinic or a water-miscible ointment base. Alternatively, the active ingredients may be formulated in a cream with an oil-in-water cream base. If desired, the aqueous phase of the cream base may include, for example, at least 30% w/w of a polyhydric alcohol, i.e. an alcohol having two or more hydroxyl groups such as propylene glycol, butane 1,3-diol, mannitol, sorbitol, glycerol and polyethylene glycol (including PEG400) and mixtures thereof. The topical formulations may desirably include a compound which enhances absorption or penetration of the active ingredient through the skin or other affected areas. Examples of such dermal penetration enhancers include dimethylsulfoxide and related analogs.

The oily phase of the emulsions of this invention may be constituted from known ingredients in a known manner. While the phase may comprise merely an emulsifier (otherwise known as an emulgent), it desirably comprises a mixture of at least one emulsifier with a fat or an oil or with both a fat and an oil. Optionally, a hydrophilic emulsifier is included together with a lipophilic emulsifier which acts as a stabilizer. It is also preferred to include both an oil and a fat. Together, the emulsifier(s) with or without stabilizer(s) make up the so-called emulsifying wax, and the wax together with the oil and fat make up the so-called emulsifying ointment base which forms the oily dispersed phase of the cream formulations.

The choice of suitable oils or fats for the formulation is based on achieving the desired cosmetic properties, since the solubility of the active compound in most oils likely to be used in pharmaceutical emulsion formulations is very low. Thus the cream should optionally be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils can be used.

Formulations suitable for topical administration to the eye also include eye drops wherein the active ingredient is dissolved or suspended in a suitable carrier, especially an aqueous solvent for the active ingredient. The active ingredient is optionally present in such formulations in a concentration of 0.5 to 20%, advantageously 0.5 to 10% particularly about 1.5% w/w. Formulations suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Formulations for rectal administration may be presented as a suppository with a suitable base comprising for example cocoa butter or a salicylate. Formulations suitable for nasal administration wherein the carrier is a solid include a coarse powder having a particle size for example in the range 20 to 500 microns (including particle sizes in a range between 20 and 500 microns in increments of 5 microns such as 30 microns, 35 microns, etc), which is administered in the manner in which snuff is taken, i.e. by rapid inhalation through the nasal passage from a container of the powder held close up to the nose. Suitable formulations wherein the carrier is a liquid, for administration as for example a nasal spray or as nasal drops, include aqueous or oily solutions of the active ingredient. Formulations suitable for aerosol administration may be prepared according to conventional methods and may be delivered with other therapeutic agents.

Formulations suitable for vaginal administration may be presented as pessaries, tampons, creams, gels, pastes, foams or spray formulations containing in addition to the active ingredient such carriers as are known in the art to be appropriate.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets of the kind previously described.

Preferred unit dosage formulations are those containing a daily dose or unit daily sub-dose, as herein above recited, or an appropriate fraction thereof, of an active ingredient.

It should be understood that in addition to the ingredients particularly mentioned above the formulations of this invention may include other agents conventional in the art having regard to the type of formulation in question, for example those suitable for oral administration may include flavoring agents.

This invention includes controlled release pharmaceutical formulations containing as active ingredient one or more compounds of the invention (“controlled release formulations”) in which the release of the active ingredient can be controlled and regulated to allow less frequency dosing or to improve the pharmacokinetic or toxicity profile of a given invention compound. Controlled release formulations adapted for oral administration in which discrete units comprising one or more compounds of the invention can be prepared according to conventional methods.

Additional ingredients may be included in order to control the duration of action of the active ingredient in the composition. Control release compositions may thus be achieved by selecting appropriate polymer carriers such as for example polyesters, polyamino acids, polyvinyl pyrrolidone, ethylene-vinyl acetate copolymers, methylcellulose, carboxymethylcellulose, protamine sulfate and the like. The rate of drug release and duration of action may also be controlled by incorporating the active ingredient into particles, e.g. microcapsules, of a polymeric substance such as hydrogels, polylactic acid, hydroxymethylcellulose, polymethyl methacrylate and the other above-described polymers. Such methods include colloid drug delivery systems like liposomes, microspheres, microemulsions, nanoparticles, nanocapsules and so on. Depending on the route of administration, the pharmaceutical composition may require protective coatings. Pharmaceutical forms suitable for injectionable use include sterile aqueous solutions or dispersions and sterile powders for the extemporaneous preparation thereof. Typical carriers for this purpose therefore include biocompatible aqueous buffers, ethanol, glycerol, propylene glycol, polyethylene glycol and the like and mixtures thereof.

In view of the fact that, when several active ingredients are used in combination, they do not necessarily bring out their joint therapeutic effect directly at the same time in the mammal to be treated, the corresponding composition may also be in the form of a medical kit or package containing the two ingredients in separate but adjacent repositories or compartments. In the latter context, each active ingredient may therefore be formulated in a way suitable for an administration route different from that of the other ingredient, e.g. one of them may be in the form of an oral or parenteral formulation whereas the other is in the form of an ampoule for intravenous injection or an aerosol.

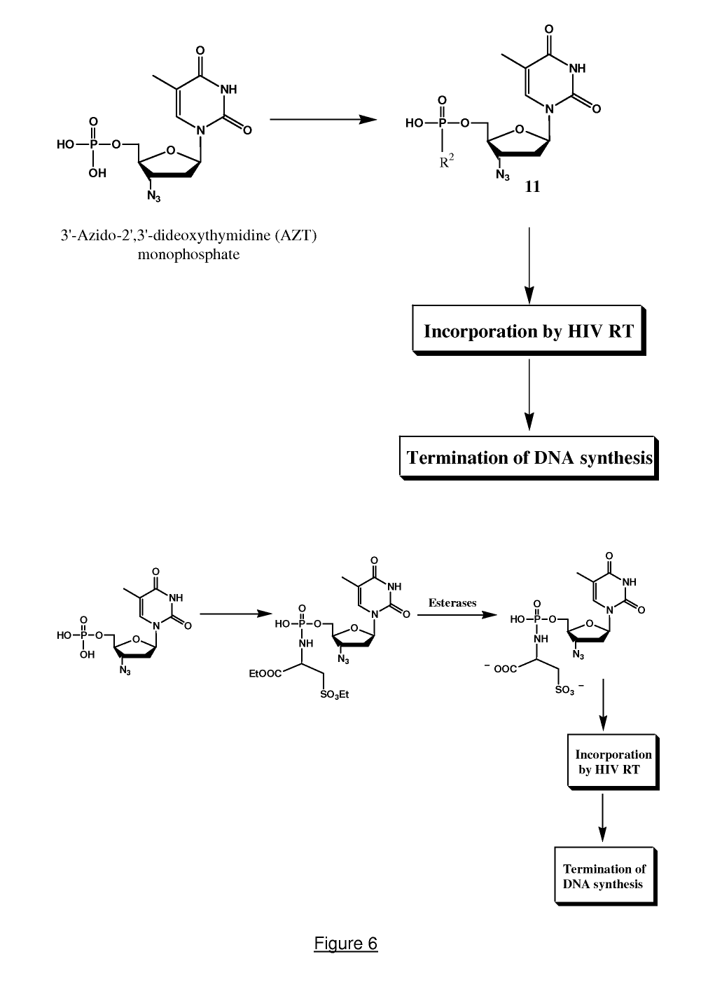

The presented invention shows that a phosphate-modified nucleoside represented by the structural formula (A) or the structural formula (I), including any one of the above-referred specific embodiments thereof, such as but not limited to 3-phosphono-L-Ala-dAMP, is successfully recognized and efficiently incorporated into a growing DNA strand by HIV RT. This means that for instance a 3-phosphono-L-Ala-phosphoramidate moiety can mimic a pyrophosphate group and behave as an easily leaving group in a nucleotidyl transfer mechanism. Such incorporation, although to a lesser extent, was also observed for L-Cys-phosphoramidates and Tau-phosphoramidates as shown in the following examples. Therefore, chain-terminating nucleotides coupled to the novel leaving groups of this invention through a phosphoramidate, phosphorothiate or phosphodiester linkage are useful for a direct inhibition of HIV RT or other retroviral polymerases as depicted in FIG. 7. Effective inhibition of HIV RT or other retroviral polymerases by a modified nucleoside of this invention requires its activation by cellular nucleoside kinases and conversion into a corresponding nucleoside triphosphate. Administration of the AZT analogue 11 as a substitute for AZT nucleoside triphosphate can therefore eliminate a requirement for kinase activation. However, it is important to assess the ability of HIV RT to recognize and insert this AZT analogue 11 with satisfactory efficiency. A potential drawback of this approach could be the charged nature of these nucleotides. As a charged molecule, these AZT analogues are not likely to pass through a cellular membrane unless active transport is involved. However, intracellular diffusion is likely facilitated by masking the negative charges of carboxylate moieties by means of esterification as shown in FIG. 7. Once a protected AZT analogue is in the cytosol, it can be transformed back to a charged, acidic form through the action of cellular esterases. FIG. 6 depicts an exemplary sulfonic ester intermediate, but this principle equally applies to other (e.g. ester) intermediates according to this invention (as shown for the AZT analogue 11). These principles of monophosphate activation and subsequent inhibition of viral polymerases, as shown in FIG. 6 for AZT, equally apply to other chain-terminating nucleotides known in the art.

The propagation of new information systems in vivo for synthetic biology purposes, requires that the system is orthogonal to the existing natural informational systems (DNA and RNA). Only in this case, it can be avoided that the natural system will become infiltrated by information from outside. This also applies to the precursors for the enzymatic synthesis of artificial nucleic acids, which means that modified building blocks used for the in vivo synthesis of artificial nucleic acids should not enter the cellular metabolic pathways, since it may lead to toxicity. One way to realize this is to develop an independent metabolic route for chemically modified polymerase substrates. This is exemplified by the new leaving groups of this invention for polymerase catalyzed nucleic acid synthesis. Preferentially the leaving group is metabolically available and can be recycled after the reaction.

Elongation results of the following examples also allow us to confirm the positive influence of a longer template overhang for the recognition and incorporation of several successive units carrying a modified leaving group of this invention, thus validating the importance of interactions between finger sub-domain residues and upstream nucleotides of the template in the HIV-RT polymerization process.

### Manufacture of the Compounds of the Invention

The synthesis of the phosphoramidate nucleosides of this invention being represented by the structural formula (I) wherein R² is represented by the structural formula (II) or the structural formula (V) may be accomplished according to the method illustrated by scheme 5 below, starting from a nucleoside monophosphate, which itself can be tailor-made by phosphorylation of a suitable nucleoside, if not yet available.

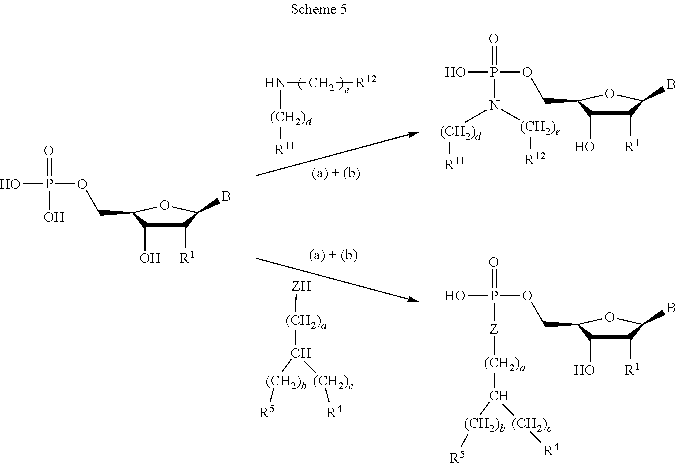

In a first step (a), the phosphate group of the 5′-mono-phosphate nucleoside is coupled with the Z-group of a reagent represented by the structural formula (G):

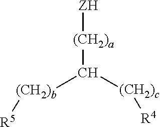

wherein Z is selected from the group consisting of O, S, NH and NR₇; and wherein R⁴, R⁵, R⁷, a, b and c are the same as defined herein-above for the structural formula (II), including any one of the above-described more specific embodiments thereof, or is coupled with the nitrogen atom of a reagent represented by the structural formula (H).

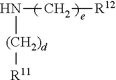

wherein R¹¹, R¹², e and d are the same as defined herein-above for the structural formula (V), including any one of the above-described more specific embodiments thereof.

With respect to formula (G), the reagent used in step (a) may be:

a 2-aminosulfocarboxylic acid (Z=NH) such as, but not limited to, cysteic acid or 2-amino-3-sulfopropanoic acid (a=b=0, c=1, R⁵=COOH, R⁴=S(O)₂OH), homocysteic acid or 2-amino-4-sulfobutanoic acid (a=b=0, c=2, R⁵=COOH, R⁴=S(O)₂OH),

an aminoalkanesulfonic acid such as, but not limited to, taurine or 2-aminoethanesulfonic acid (Z=NH, a=b=0, c=1, R⁵=H, R⁴=S(O)₂OH), homotaurine or 3-aminopropanesulfonic acid (Z=NH, a=b=0, c=2, R⁵=H, R⁴=S(O)₂OH), aminomethanesulfonic acid (Z=NH, a=b=c=O, R⁵=H, R⁴=S(O)₂OH), 4-aminobutanesulfonic acid (Z=NH, a=b=0, c=3, R⁵=H, R⁴=S(O)₂OH), N-methyltaurine or 2-(methylamino)ethanesulfonic acid (Z=NR⁷, a=b=0, c=1, R⁵=H, R⁴=S(O)₂OH and R′=methyl), N-ethyltaurine or 2-(ethylamino)ethanesulfonic acid (Z=NR⁷, a=b=0, c=1, R⁵=H, R⁴=S(O)₂OH and R⁷=ethyl), N-propyltaurine or 2-(propylamino)ethanesulfonic acid (Z=NR⁷, a=b=0, c=1, R⁵=H, R⁴=S(O)₂OH and R⁷=propyl), N-butyltaurine or 2-(butylamino)ethanesulfonic acid (Z=NR⁷, a=b=0, c=1, R⁵=H, R⁴=S(O)₂OH and R⁷=butyl), N-phenyltaurine or 2-(phenylamino)ethanesulfonic acid (Z=NR⁷, a=b=0, c=1, R⁵=H, R⁴=S(O)₂OH and R⁷=phenyl), N-benzyltaurine or 2-(benzylamino)ethanesulfonic acid (Z=NR⁷, a=b=0, c=1, R⁵=H, R⁴=S(O)₂OH and R⁷=benzyl), N-cyclohexyltaurine or 2-(cyclohexylamino)ethanesulfonic acid (Z=NR⁷, a=b=0, c=1, R⁵=H, R⁴=S(O)₂OH and R⁷=cyclohexyl);

an ω-hydroxy alkanesulfonic acid (a=b=0, R⁵=hydrogen) such as, but not limited to, hydroxymethanesulfonic acid (c=0), 2-hydroxymethanesulfonic acid or isethionic acid (c=1), 3-hydroxypropanesulfonic acid (c=2) or 4-hydroxybutanesulfonic acid (c=3);

an ω-hydroxy alkanephosphonic acid (a=b=0, R⁵=hydrogen) such as, but not limited to, hydroxymethanephosphonic acid (c=0), 2-hydroxymethanephosphonic acid (c=1) or 3-hydroxypropanephosphonic acid (c=2).

The reagent used in step (a) may also be a non-natural amino acid as described for instance in U.S. Patent application publication No. 2010/061936, the content of which is incorporated herein by reference. This will afford modified nucleosides represented by the structural formula (A) or the structural formula (I) wherein R₂ is represented by the structural formula (II), wherein Z is NH, a=b=0, c is 1 and R⁵ is COOH, and wherein R⁴ is selected from the group consisting of C₆H₅—OP(O)(OH)₂ wherein said C₆H₅ (phenyl) is substituted with fluoromethyl or difluoromethyl; C₆H₅—CHXP(O)(OH)₂; C₆H₅-QS(O)₂(CH═CH₂); C₆H₅-QV wherein said C₆H₅ (phenyl) is substituted with oxiran-2-yl or CH═CH₂; C₆H₅—C(═CH₂)V; CHXV and C(═CH₂)V;

X is chloro or bromo;

Q is a linking moiety selected from the group consisting of O, CH₂, (CH₂)₂ and CF₂;

V is selected from the group consisting of P(O)(OH)₂, S(O)₂(OH), SO₂NH₂, SO₂CH₃ and SO₂CF₃.

In particular it may be a non-natural amino acid selected from the group consisting of:
2-amino-3-(3-(fluoromethyl)-4-(phosphonooxy)phenyl)propanoic acid,
2-amino-3-(3-(difluoromethyl)-4-(phosphonooxy)phenyl)propanoic acid;
2-amino-3-(4-(bromo(phosphono)methyl)phenyl)propanoic acid,
2-amino-3-(4-(chloro(phosphono)methyl)phenyl)propanoic acid,
2-amino-3-(4-(1-phosphonovinyl)phenyl)propanoic acid,
2-amino-3-(4-(1-sulfovinyl)phenyl)propanoic acid,
2-amino-3-(4-(1-sulfamoylvinyl)phenyl)propanoic acid,
2-amino-3-(4-(1-(methylsulfonyl)vinyl)phenyl)propanoic acid,
2-amino-3-(4-(1-(trifluoromethylsulfonyl)vinyl)phenyl)propanoic acid,
2-amino-3-(4-(phosphonooxy)-3-vinylphenyl)propanoic acid,
2-amino-3-(4-(sulfooxy)-3-vinylphenyl)propanoic acid,
2-amino-3-(4-(sulfamoyloxy)-3-vinylphenyl)propanoic acid,
2-amino-3-(4-(methylsulfonyloxy)-3-vinylphenyl)propanoic acid,
2-amino-3-(4-(trifluoromethylsulfonyloxy)-3-vinylphenyl)propanoic acid,
2-amino-3-(4-(phosphonoamino)-3-vinylphenyl)propanoic acid,
2-amino-3-(4-(sulfoamino)-3-vinylphenyl)propanoic acid,
2-amino-3-(4-(sulfamoylamino)-3-vinylphenyl)propanoic acid,
2-amino-3-(4-(methylsulfonamido)-3-vinylphenyl)propanoic acid,
2-amino-3-(4-(trifluoromethylsulfonamido)-3-vinylphenyl)propanoic acid,
2-amino-3-(4-phosphono-3-vinylphenyl)propanoic acid,
2-amino-3-(4-sulfo-3-vinylphenyl)propanoic acid,
2-amino-3-(4-sulfamoyl-3-vinylphenyl)propanoic acid,
2-amino-3-(4-(methylsulfonyl)-3-vinylphenyl)propanoic acid,
2-amino-3-(4-(trifluoromethylsulfonyl)-3-vinylphenyl)propanoic acid,
2-amino-3-(4-(phosphonomethyl)-3-vinylphenyl)propanoic acid,
2-amino-3-(4-(sulfomethyl)-3-vinylphenyl)propanoic acid,
2-amino-3-(4-(sulfamoylmethyl)-3-vinylphenyl)propanoic acid,
2-amino-3-(4-(methylsulfonylmethyl)-3-vinylphenyl)propanoic acid,
2-amino-3-(4-(trifluoromethylsulfonylmethyl)-3-vinylphenyl)propanoic acid,
2-amino-3-(4-(difluoro(phosphono)methyl)-3-vinylphenyl)propanoic acid,
2-amino-3-(4-(difluoro(sulfo)methyl)-3-vinylphenyl)propanoic acid,
2-amino-3-(4-(difluoro(sulfamoyl)methyl)-3-vinylphenyl)propanoic acid,
2-amino-3-(4-(difluoro(methylsulfonyl)methyl)-3-vinylphenyl)propanoic acid,
2-amino-3-(4-(difluoro(trifluoromethylsulfonyl)methyl)-3-vinylphenyl)propanoic acid,
2-amino-3-(3-(oxiran-2-yl)-4-(phosphonooxy)phenyl)propanoic acid,
2-amino-3-(3-(oxiran-2-yl)-4-(sulfooxy)phenyl)propanoic acid,
2-amino-3-(3-(oxiran-2-yl)-4-(sulfamoyloxy)phenyl)propanoic acid,
2-amino-3-(4-(methylsulfonyloxy)-3-(oxiran-2-yl)phenyl)propanoic acid,
2-amino-3-(3-(oxiran-2-yl)-4-(trifluoromethylsulfonyloxy)phenyl)propanoic acid,
2-amino-3-(3-(oxiran-2-yl)-4-(phosphonoamino)phenyl)propanoic acid,
2-amino-3-(3-(oxiran-2-yl)-4-(sulfoamino)phenyl)propanoic acid,
2-amino-3-(3-(oxiran-2-yl)-4-(sulfamoylamino)phenyl)propanoic acid,
2-amino-3-(4-(methylsulfonamido)-3-(oxiran-2-yl)phenyl)propanoic acid,
2-amino-3-(3-(oxiran-2-yl)-4-(trifluoromethylsulfonamido)phenyl)propanoic acid,
2-amino-3-(3-(oxiran-2-yl)-4-phosphonophenyl)propanoic acid,
2-amino-3-(3-(oxiran-2-yl)-4-sulfophenyl)propanoic acid,
2-amino-3-(3-(oxiran-2-yl)-4-sulfamoylphenyl)propanoic acid,
2-amino-3-(4-(methylsulfonyl)-3-(oxiran-2-yl)phenyl)propanoic acid,
2-amino-3-(3-(oxiran-2-yl)-4-(trifluoromethylsulfonyl)phenyl)propanoic acid,
2-amino-3-(3-(oxiran-2-yl)-4-(phosphonomethyl)phenyl)propanoic acid,
2-amino-3-(3-(oxiran-2-yl)-4-(sulfomethyl)phenyl)propanoic acid,
2-amino-3-(3-(oxiran-2-yl)-4-(sulfamoylmethyl)phenyl)propanoic acid,
2-amino-3-(4-(methylsulfonylmethyl)-3-(oxiran-2-yl)phenyl)propanoic acid,
2-amino-3-(3-(oxiran-2-yl)-4-((trifluoromethylsulfonyl)methyl)phenyl)propanoic acid,
2-amino-3-(4-(difluoro(phosphono)methyl)-3-(oxiran-2-yl)phenyl)propanoic acid,
2-amino-3-(4-(difluoro(sulfo)methyl)-3-(oxiran-2-yl)phenyl)propanoic acid,
2-amino-3-(4-(difluoro(sulfamoyl)methyl)-3-(oxiran-2-yl)phenyl)propanoic acid,
2-amino-3-(4-(difluoro(methylsulfonyl)methyl)-3-(oxiran-2-yl)phenyl)propanoic acid,
2-amino-3-(4-(difluoro(trifluoromethylsulfonyl)methyl)-3-(oxiran-2-yl)phenyl)propanoic acid,
2-amino-3-(4-(vinylsulfonyloxy)phenyl)propanoic acid,
2-amino-3-(4-(vinylsulfonamido)phenyl)propanoic acid,
2-amino-3-(4-(vinylsulfonyl)phenyl)propanoic acid,
2-amino-3-(4-(vinylsulfonylmethyl)phenyl)propanoic acid,
2-amino-3-(4-(difluoro(vinylsulfonyl)methyl)phenyl)propanoic acid,
2-amino-4-bronno-4-phosphonobutanoic acid,
2-amino-4-bromo-4-sulfobutanoic acid,
2-amino-4-bronno-4-sulfamoylbutanoic acid,
2-amino-4-bronno-4-(methylsulfonyl)butanoic acid,
2-amino-4-bronno-4-(trifluoromethylsulfonyl)butanoic acid,
2-amino-4-chloro-4-phosphonobutanoic acid,
2-amino-4-chloro-4-sulfobutanoic acid,
2-amino-4-chloro-4-sulfamoylbutanoic acid,
2-amino-4-chloro-4-(methylsulfonyl)butanoic acid,
2-amino-4-chloro-4-(trifluoromethylsulfonyl)butanoic acid,
2-amino-4-phosphonopent-4-enoic acid,
2-amino-4-sulfopent-4-enoic acid,
2-amino-4-sulfamoylpent-4-enoic acid,
2-amino-4-(methylsulfonyl)pent-4-enoic acid,
2-amino-4-(trifluoromethylsulfonyl)pent-4-enoic acid,
2-amino-3-(vinylsulfonyloxy)propanoic acid,
2-amino-3-(vinylsulfonamido)propanoic acid,
2-amino-3-(vinylsulfonyl)propanoic acid,
2-amino-4-(vinylsulfonyl)butanoic acid, and
2-amino-4,4-difluoro-4-(vinylsulfonyl)butanoic acid.

Said coupling reaction results in the formation of a phosphate ester (when Z=O), phosphate thioester or phosphorothioate (when Z=S), or phosphoramide (when Z=NH, or NCH₃; or when using a reagent (H)). Said coupling reaction may be performed using any coupling agent (also referred to as dehydrating agent) known in the art for esterification, thioesterification or amide formation, in particular using a carbodiimide coupling agent, more particularly using dicyclohexylcarbodiimide (DCC) or N-ethyl-N′-(3-dimethylaminopropyl)-carbodiimide hydrochloride (EDAC). The coupling reaction may be performed in the presence of a suitable solvent at any temperature between room temperature and reflux temperature of said solvent. Depending on the nature of R⁴, R⁵, R¹¹ or R¹², additional acid, hydroxyl or amine functionalities in the reagent (G) or (H) may be transiently protected to prevent these functionalities from interfering with the condensation reaction between the phosphate acid and the Z or N atom from respectively the reagent (G) or (H). Therefore, this synthetic route provides for an optional subsequent step (b) of deprotecting such functionalities. This deprotection step (b) can be carried out by means of any method known in the art, e.g. with potassium carbonate in a methanol-water solution.

In the above scheme 5, B can be a pyrimidine base according to formula (C) or a purine base according to formula (D) thus illustrating a synthetic route for the synthesis of pyrimidine and purine derived compounds according to the present invention respectively.

Alternatively, the compounds represented by the structural formula (A) or the structural formula (I), or any one of the above-described specific embodiments thereof, may be obtained according to synthetic procedures illustrated by any one of schemes 1, 2, 3 or 4 herein above.

The following examples are provided for illustrative purpose only, and should not be considered as limiting the scope of the present invention. The synthesis of the esters of the phosphate-modified nucleosides of the invention was accomplished along the general principles of the method described by Wagner et al. in Mini-Rev. Med. Chem. (2004) 4:409, starting from a nucleoside monophosphate. Deprotection of the esters was carried out with potassium carbonate in methanol-water solution.

In these examples, the following analytical methods and materials were used. For all reactions, analytical grade solvents were used. A Bruker Avance II 600 MHz or 500 MHz spectrometer and a Bruker Avance 300 MHz apparatus were used for ¹H NMR, ¹³C NMR and ³¹P NMR. For sake of clarity of the NMR signal assignment, sugar protons and carbons are numbered with a prime. ³¹P NMR chemical shifts are referenced to an external 85% H₃PO₄ standard (δ=0.000 ppm). Accurate mass spectra were recorded on a Fourier transform ion-cyclotron resonance (FTICR) mass spectrometer, apex-Qe (Bruker Daltonics, Bremen, Germany) with a passively shielded 9.4 Tesla superconducting magnet equipped with an Apollo 2 CombiSource (Bruker Daltonics, Bremen, Germany). Tuning in positive electrospray mode to resolution of 95000 (at m/z 400) and calibration (from m/z 100 to 1500) were performed using poly-DL-alanine (Sigma-Aldrich, St. Louis, Mo.). Spray voltage was set to 4000 V, capillary temperature 240° C. Samples were infused in a water:acetonitrile (1:1) mixture with a flow rate of 180 pUh. 32 scans of 512 k datapoints were acquired and averaged. For the acquisition and processing software ApexControl 1.0 and DataAnalysis 3.4 (Bruker Daltonics, Bremen, Germany) were used respectively. Precoated aluminum sheets (MN ALUGRAM SIL G/UV₂₅₄20×20 cm) were used for TLC; the spots were examined with UV light. Column chromatography was performed on ICN silica gel 63-200, 60 Å. Preparative HPLC was performed on waters 1525-2487 system using Prep C18 5 μm column 19×150 mm at the flow rate of 3 mL/min by a gradient elution of acetonitrile and 50 mM triethylammonium bicarbonate buffer.

Oligodeoxyribonucleotides P1, T1, T2 and T3 were purchased from Sigma Genosys. The concentrations were determined with a Varian Cary-300-Bio UV Spectrophotometer. The lyophilized oligonucleotides were dissolved in diethylpyrocarbonate (DEPC)-treated water and stored at −20° C. The primer oligonucleotides were 5′-³³P-labeled with 5′-[γ³³P]-ATP (Perkin Elmer) using T4 polynucleotide kinase (New England Biolabs) according to standard procedures. The labeled oligonucleotide was further purified using Illustra™ Microspin™ G-25 columns (GE Healthcare).

DNA polymerase reactions: end-labeled primer was annealed to its template by combining primer and template in a molar ratio of 1:2 and heating the mixture to 70° C. for 10 minutes followed by slow cooling to room temperature over a period of 1.5 hour. For the incorporation of a modified nucleoside, a series of 20 μL-batch reactions was performed with the enzyme HIV-1 RT (Ambion, 10 U/μL stock soln, specific activity 8.095 Wring, concentration 1.2 mg/mL). The final mixture contained 125 nM primer template complex, RT buffer (250 mM Tris.HCl, 250 mM KCl, 50 mM MgCl₂, 2.5 mM spermidine, 50 mM dithiothreitol (DTT); pH 8.3), 0.025 U/μL HIV-1 RT, and different concentrations of the phosphoramidate to be tested (1 mM, 500 μM, 200 μM and 100 μM).

Electrophoresis: all polymerase reaction aliquots (2.5 μL) were quenched by the addition of 10 μL of loading buffer (90% formamide, 0.05% bromophenol blue, 0.05% xylene cyanol and 50 mM ethylenediaminetetraacetic acid (EDTA)). Samples were heated at 85° C. for 5 minutes prior to analysis by electrophoresis for 2.5 hours at 2000 V on a 30 cm×40 cm×0.4 mm 20% (19:1 mono:bis) denaturing gel in the presence of a 100 mM Tris-borate, 2.5 mM EDTA buffer; pH 8.3. Products were visualized by phosphorimaging. The amount of radioactivity in the bands corresponding to the products of enzymatic reactions was determined by using the imaging device Cyclone@ and the Optiquant image analysis software (Perkin Elmer).

### Example 1

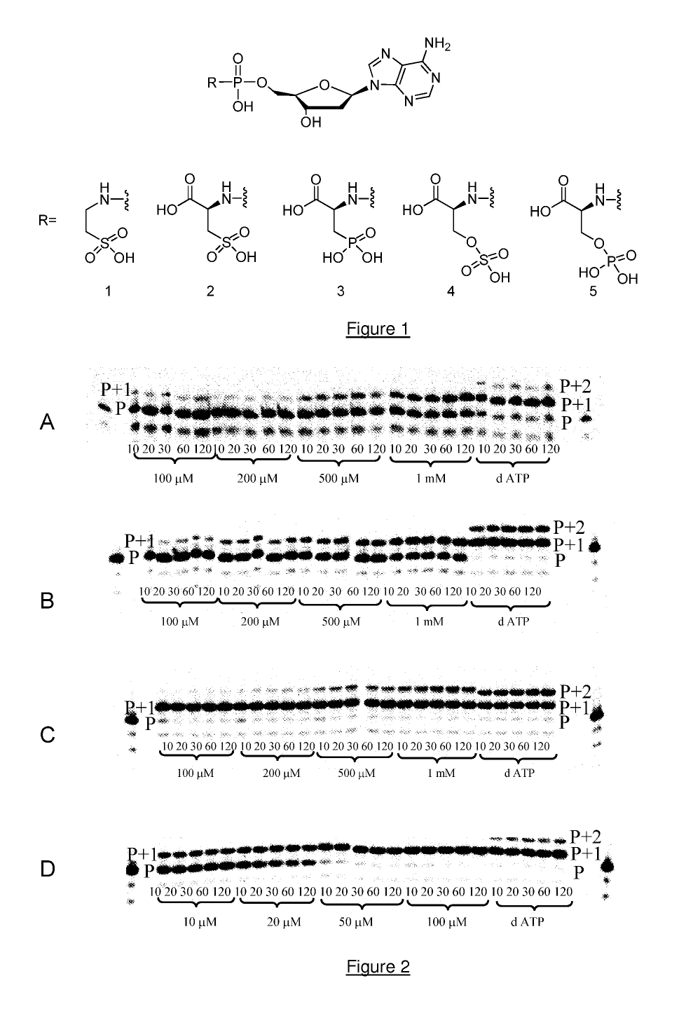

### Synthesis of Compounds 1 and 2 (Structures Shown in FIG. 1)

The synthesis of Tau-dAMP (1) or L-Cys-dAMP (2) is shown in scheme 6. Tau-dAMP (1) or L-Cys-dAMP (2) were synthesized from deoxyadenosine monophosphate (dAMP) and taurine or L-cysteic acid ester by an N-Ethyl-N′-(3-dimethylaminopropyl)-carbodiimide hydrochloride (EDAC) mediated coupling, followed by a deprotection with base. The coupling and deprotection of taurine and L-cysteic acid was done in a one-pot reaction.

The coupling reaction was first performed according to the general teaching of Huang et al in RNA (2003) 9:1562-1570 (at room temperature in water, for 2 hours in the presence of EDAC as the coupling agent). However, in order to avoid the presence of the side products, this methodoly was modified by carrying the reaction in a one-pot process using a ratio of 3.3:1 (EDAC/dAMP) and, as a result, the yield of the desired compounds increased considerably.

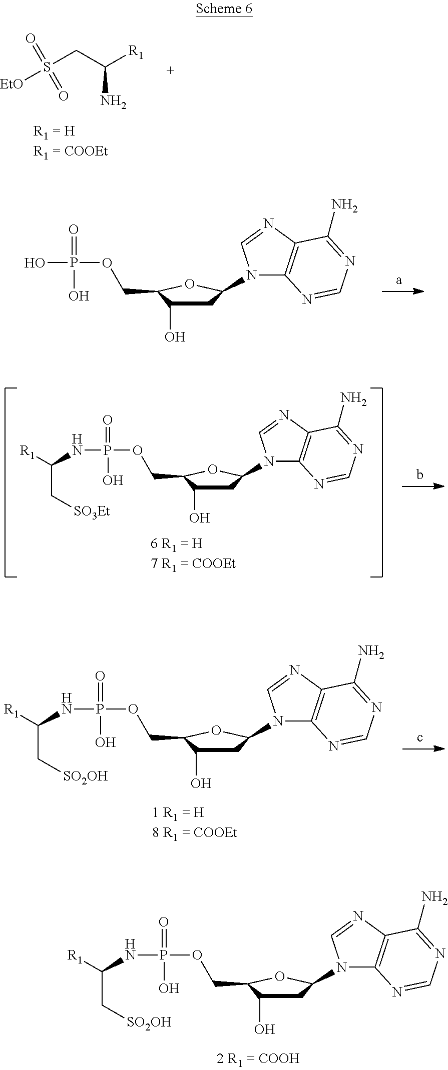

Reagents and conditions were: (a) EDAC, H₂O, room temperature, 2-5 hours, under argon atmosphere; (b) 1.4M K₂CO₃ in MeOH/H₂O=1:1, room temperature, 2 hours, under argon atmosphere; (c) 0.4M NaOH in MeOH/H₂O=1:1, room temperature, 15 hours, under argon atmosphere.

The synthesis of 3-phosphono-L-Ala-dAMP (3), O-phospho-L-Ser-dAMP (4) and O-sulfonato-L-Ser-dAMP (5) is described in scheme 7. 3-phosphono-L-Ala-dAMP (3), O-phospho-L-Ser-dAMP (4) and O-sulfonato-L-Ser-dAMP (5) were synthesized from deoxyadenosine-5′-phosphorimidazolide (ImpA) and L-alanine-3-phosphono acid, L-serine-O-sulfate and L-serine-O-phosphate respectively, by a one-step reaction.

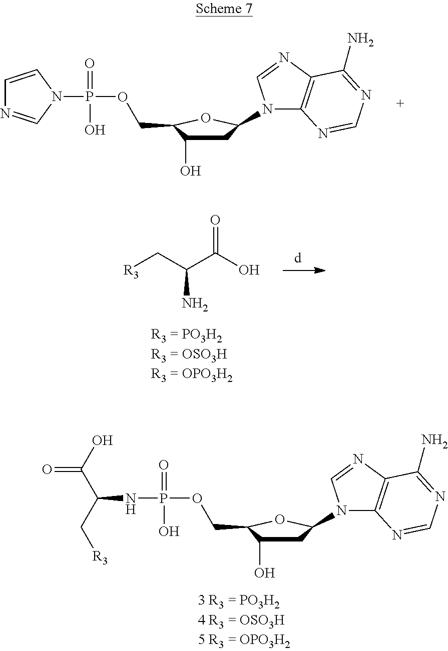

Reagents and conditions were: (d) N-ethylmorpholine, H₂O, room temperature, 3 days, under argon atomsphere.

When using the reaction conditions of Sawai (reaction of deoxyadenosine-5′-phosphorimidazolide (ImpA) with glycolic acid or lactic acid) for the synthesis of the phosphoramidates 3-5, these compounds were found difficult to purify. This problem was solved by modifying the reaction by using more N-ethylmorpholine as a base and without adding any metal ions. The phosphoramidate products were first purified by column chromatography followed by preparative HPLC. In this one-step reaction, the phosphoramidates 3-5 were directly obtained from deoxyadenosine-5′-phosphorimidazolide (ImpA) and related amino acid derivatives by nucleophilic substitution.

### 2′-deoxyadenosine-5′-taurine phosphoramidate (compound 1)

2′-deoxyadenosine-5′-monophosphoric acid hydrate (40 mg, 0.11 mmol) and taurine ethyl ester (81 mg, 0.36 mmol) were suspended in 1 mL water and stirred for 5 minutes under Argon. Then N-Ethyl-N′-(3-dimethylaminopropyl)-carbodiimide hydrochloride (EDAC, 69 mg, 0.36 mmol) was added to the suspension and the coupling reaction was continued under stirring at room temperature. After 2 hours, 1.4M K₂CO₃ (MeOH/H₂O=1:1) 1.2 mL was added to the coupling reaction mixture and a deprotection reaction of ethyl ester was immediately carried out at room temperature while stirring under argon atmosphere. Evolution of the reaction was monitored by TLC (CHCl₃/MeOH/H₂O 5:4:0.5) and ³¹P NMR until the disappearance of the ester intermediate. The reaction mixture was neutralized by addition of 1 M TEAA. The solvent was evaporated to dryness in vacuum. The residue was purified by silica column chromatography eluting with i-PrOH/NH₃/:H₂O 12:1:1, 10:1:1 to 9:1:1 to yield compound 1 (30 mg, 57%) as white solid.

### 2′-deoxyadenosine-5′-(3-phosphono-L-alanine) phosphoramidate (compound 3)

Deoxyadenosine-5′-phosphorimidazolide was first synthesized as follows. A mixture of dAMP (100 mg, 0.3 mmole), dithiopyridine (211 mg, 0.96 mmoles), triphenylphosphine (327 mg, 0.96 mmole) and imidazole (327 mg, 4.8 mmoles) was dried under high vacuum for 30 minutes. Subsequent dissolution in anhydrous DMSO (3.5 mL) under argon atmosphere afforded a clear yellow solution. Triethylamine (95% L, 0.7 mmole) was added via a syringe and the resulting solution stirred at room temperature for 4 hours. It was then dropped in a −20° C. 0.1 M sodium iodide solution in dry acetone and allowed to precipitate for 15 minutes. Filtration of the resulting suspension and repeated washing thereof with cold dry acetone afforded a white solid. Further drying on POCl₃ under HV afforded quantitative yield of dAMP-imidazolide (110 mg, 96%) which was characterized as follows:

¹H NMR (300 MHz, D₂O, 5° C.): δ8.21 (s, 1H, H₈), 8.20 (s, 1H, H₂), 7.7 (s, 1H, Hₗₘ), 7.0 (s, 1H, Hₗₘ), 6.8 (s, 1H, Hₗₘ), 6.4 (apparent t, ³J_{H1′-H2′}=6.41 Hz, 1H, H_{1′}), 4.6 (m, 1H, H_{3′}), 4.2 (m, 1H, H_{4′}), 4.0 (m, 2H, H_{5′}), 2.9 (m, 1H, H_{2′}), 2.6 (m, 1H, H_{2′}) ppm;

³¹P NMR (121 MHz, D₂O): δ −8.01 ppm; and

High res. MS (ESI): calculated for C13H16N7O5P1: 381.0951. found: 380.0869 (negative mode).

In a 25 ml flask, L-Alanine, 3-phosphono acid HCl salt (60 mg, 0.29 mmol) was stirred in 0.5 mL N-ethyl morpholine for 5 minutes at room temperature, then deoxyadenosine-5′-phosphorimidazolide (240 mg, 0.63 mmol) and 6 mL 0.2 M N-ethyl morpholine buffer (pH=7.5) were added into the flask. The reaction mixture was continued to stir for 3 days at room temperature under Argon (Ar). The reaction process was checked by TLC (i-PrOH/NH₃/H₂O=7:1:2) and ³¹P NMR. When there were no more products formed, the reaction mixture was concentrated to dryness in vacuum (30° C. bath). The residue was purified by silica column chromatography eluting with i-PrOH/NH₃/H₂O=20:1:1, 9:1:1, 7:1:1 to 4.5:1:1 and yielded crude white solid (32 mg, 23.8%). The product was further purified by preparative HPLC with a gradient of CH₃CN in 50 mm triethylammonium bicarbonate buffer to yield compound 3 (10 mg, 31.3% yield)

### 2′-Deoxyadenosine-5′-taurine phosphoramidate (1)

¹H NMR (300 MHz, D₂O): δ 8.43 (s, 1H), 8.18 (s, 1H), 6.46 (m, 1H, H-1′), 4.71 (m, 1H, H-3′), 4.26 (m, 1H, H-4′), 3.97 (m, 2H, H-5′, CH₂), 3.16 (m, 2H, —CH₂CH₂SO₃H), 2.93 (m, 2H, —CH₂CH₂SO₃H), 2.85 (m, 1H, H-2′), and 2.60 (m, 1H, H-2′) ppm;

¹³C NMR (75 MHz, D₂O): δ 155.25, 152.35, 148.57, 139.85, 118.49, 85.95 (d, J (C, P)=8.85, C-4′), 83.66 (C-1′), 71.29 (C-3′), 64.02 (d, J (C, P)=5.09, C-5′), 52.25 (d, J (C, P)=6.34, —CH₂CH₂SO₃H), 39.00 (C-2′), and 36.93 (—CH₂CH₂SO₃H);

³¹P NMR (121 MHz, D₂O): δ 8.16;

HRMS for C₁₂H₁₉N₆O₈PS (M−H)⁻ calcd: 437.0650. found: 437.0634.

### 2′-Deoxyadenosine-5′-(L-cysteic acid) phosphoramidate (2)

¹H NMR (500 MHz, D₂O): δ 8.50 (s, 1H), 8.24 (s, 1H), 6.51 (m, 1H, H-1′), 4.72 (m, 1H, H-3′), 4.26 (m, 1H, H-4′), 3.99 (m, 2H, H-5′, CH₂), 3.87 (m, 1H, —CHCOOH), 3.19 (m, 2H, —CH₂SO₃H), 2.82 (m, 1H, H-2′), and 2.58 (m, 1H, H-2′) ppm;

¹³C NMR (125 MHz, D₂O): δ 178.61 (d, J (C, P)=6.50, —COOH), 155.14, 152.22, 148.35, 139.57, 118.20, 85.73 (d, J (C, P)=15.09, C-4′), 83.20 (C-1′), 70.95 (C-3′), 63.57 (d, J (C, P)=7.53, C-5′), 54.38 (d, J (C, P)=10.00, —CH₂SO₃H), 53.69 (—CHCOOH), and 38.63 (C-2′);

³¹P NMR (202 MHz, D₂O): δ 6.35; and

HRMS for C₁₃H₁₉N₆O₁₀PS (M−H)⁻ calcd: 481.0548. found: 481.0568

### 2′-Deoxyadenosine-5′-(3-phosphono-L-alanine) phosphoramidate (3)

¹H NMR (600 MHz, D₂O, 4° C.,): δ 8.39 (d, J=12 Hz, 1H), 8.11 (s, 1H), 6.39 (m, 1H, H-1′), 4.58 (m, 1H, H-3′), 4.14 (m, 1H, H-4′), 3.85 (m, 2H, H-5′, CH₂), 3.64 (m, 1H, —CHCOOH), 2.94 (m, 1H, —CH₂PO₃H₂), 2.85 (m, 1H, —CH₂PO₃H₂), 2.71 (m, 1H, H-2′), and 2.45 (m, 1H, H-2′) ppm;

¹³C NMR (125 MHz, D₂O, 4° C.,: δ 180.99 (d, J (C, P)=12.96 Hz, —COOH), 155.18, 152.17, 148.36, 139.55 (d, J (C, P)=18.68 Hz), 118.17, 85.44 (dd, J₁ (C, P)=8.67 Hz, J₂ (C, P)=21.95 Hz, C-4′), 83.27 (d, J (C, P)=2.67 Hz, C-1′), 71.13 (d, J₁ (C, P)=5.22 Hz, C-3′), 63.74 (C-5′), 52.79 (—CHCOOH), 42.41 (—CH₂PO₃H₂), and 38.53 (d, J (C, P)=11.97, C-2′);

³¹P NMR (121.2 MHz, D₂O): δ 21.13, 7.13; and

HRMS for C₁₃H₂₀N₆O₁₀P₂ (M−H)⁻ calcd: 481.0643. found: 481.0654

### 2′-Deoxyadenosine-5′-(O-sulfonato-L-serine) phosphoramidate (4)

¹H NMR (300 MHz, D₂O): δ 8.50 (s, 1H), 8.25 (s, 1H), 6.51 (m, 1H, H-1′), 4.71 (m, 1H, H-3′), 4.28 (m, 1H, H-4′), 4.16 (m, 1H, —CH₂OSO₃H), 4.07 (m, 1H, —CH₂OSO₃H), 4.00 (m, 2H, H-5′, CH₂), 3.76 (m, 1H, —CHCOOH), 2.83 (m, 1H, H-2′), and 2.60 (m, 1H, H-2′) ppm;

¹³C NMR (75 MHz, D₂O): δ 177.08 (—COOH), 154.38, 151.08, 148.33, 140.01, 118.28, 85.90 (d, J (C, P)=9.23, C-4′), 83.54 (C-1′), 71.22 (C-3′), 70.74 (—CH₂OSO₃H), 63.80 (d, J (C, P)=5.09, C-5′), 55.58 (—CHCOOH), and 38.83 (C-2′);

³¹P NMR (121.2 MHz, D₂O): δ 6.54; and

HRMS for C₁₃H₁₉N₆O₁₁P₁S₁ (M calcd: 497.0497. found: 497.0471

### 2′-Deoxyadenosine-5′-(O-phospho-L-serine) phosphoramidate (5)

¹H NMR (300 MHz, D₂O): δ 8.47 (s, 1H), 8.20 (s, 1H), 6.47 (m, 1H, H-1′), 4.64 (m, 1H, H-3′), 4.24 (m, 1H, H-4′), 3.99 (m, 1H, —CH₂OPO₃H₂), 3.94 (m, 2H, H-5′, CH₂), 3.88 (m, 1H, —CH₂OPO₃H₂), 3.64 (m, 1H, —CHCOOH), 2.77 (m, 1H, H-2′), and 2.54 (m, 1H, H-2′) ppm;

¹³C NMR (125 MHz, D₂O): δ 171.02 (—COOH), 153.72, 150.29, 147.85, 139.66, 117.77, 85.64 (d, C-4′, J (C, P)=8.95), 83.16 (C-1′), 70.87 (C-3′), 67.41 (—CH₂OPO₃H₂), 63.46 (d, C-5′, J (C, P)=4.63), 56.04 (d, —CHCOOH, J (C, P)=8.75), and 38.53 (C-2′);

³¹P NMR (121.2 MHz, D₂O): δ 6.76, 0.28; and

HRMS for C₁₃H₂₀N₆O₁₁P₂ (M−H)⁻ calcd: 497.0592. found: 497.0585

### 2′-Deoxyadenosine-5′-(ethyl L-cysteate) phosphoramidate (intermediate for 2)

¹H NMR (500 MHz, D₂O): δ 8.42 (s, 1H), 8.24 (s, 1H), 6.49 (m, 1H, H-1′), 4.80 (m, 1H, H-3′), 4.25 (m, 1H, H-4′), 4.05 (m, 2H, —OCH₂CH₃), 4.03 (m, 2H, H-5′, CH₂), 3.85 (m, 1H, —CHCOOH), 3.09 (m, 2H, —CH₂SO₂OH), 2.86 (m, 1H, H-2′), and 2.62 (m, 1H, H-2′) ppm,

¹³C NMR (125 MHz, D₂O): δ 173.52, 155.22, 152.37, 148.40, 139.52, 118.27, 85.60 (d, J (C, P)=16.16, C-4′), 83.26 (C-1′), 70.90 (C-3′), 63.74 (d, J (C, P)=8.08, C-5′)), 62.02 (—OCH₂CH₃), 53.22 (d, J (C, P)=8.08, —CH₂SO₃H), 51.13 (—CHCOOEt), 38.45 (C-2′), and 12.75 (—OCH₂CH₃);

³¹P NMR (202 MHz, D₂O): δ 5.50;

HRMS for C₁₅H₂₃N₆O₁₀PS (M−H)⁻ calcd: 509.0861. found: 509.0884.

### Example 2

### Single Nucleotide Incorporation of HIV-1 RT)

HIV-1 Reverse Transcriptase serves, in the HIV-1 viral replication process, as catalyst and uses deoxynucleotides as substrates. This polymerase is error-prone and thus has a high mutation rate. Previous experiments carried out with L-aspartic acid phosphoramidate of dAMP demonstrated that this amino acid was an acceptable leaving group for the polymerase in the nucleotidyl insertion process. Incorporation results were lower using the enantiomeric D-Asp leaving group.

Here, the ability of phosphoramidate analogues as substrate for HIV-1 RT was investigated by the gel-based single nucleotide incorporation assay. The natural nucleoside triphosphate (dATP) was used as reference.

Among the five phosphoramidate analogues (1-5), 3-phosphono-L-Ala-dAMP (3) showed the best incorporation efficiency (FIG. 3). In single nucleotide incorporation by HIV-1 RT, it resulted in 94.6% conversion to a P+1 strand within 60 minutes at 50 μM (phosphoramidate analogue concentration). Incorporation of 3-phosphono-L-Ala-dAMP (3) (27.1%) was also observed when the phosphoramidate substrate concentration was 10 μM.

At the same conditions, Tau-dAMP (1), or L-Cys-dAMP (2), O-sulfonato-L-Ser-dAMP (4), O-phospho-L-Ser-dAMP (5) were less efficient, showing only 54.7%, 63.1%, 8.1%, and 19.4% conversion to a P+1 strand within 60 minutes at 1 mM, respectively (Table 1). Comparing with L-Cys-dAMP (2), 3-phosphono-L-Ala-dAMP (3) was 10.7-fold more efficient, suggesting that a phosphonate group may be better accommodated in the active site of HIV RT than a sulfonate group. The phosphonate group also has an additional charge which may have a beneficial effect on the catalytic process. O-sulfonato-L-Ser-dAMP (4) and O-phospho-L-Ser-dAMP (5) were much less good substrates for HIV-1 RT in the polymerase reaction. Additionally, the sulfate residue of 4 is unstable and easily hydrolyzed into Ser-dAMP (already known to be a poor substrate).

TABLE 1

| 3-phosphono-L-Ala- | | | | | | |
|---|---|---|---|---|---|---|
| dAMP | | Time points (min) | | | | |
| (compound 3) | | 10 | 20 | 30 | 60 | 120 |
| 50 μM | (% of P + 1) | 84.5 | 89.9 | 92.6 | 94.6 | 95.9 |
| 100 μM | | 94.8 | 97.7 | 97.6 | 97.9 | 97.5 |
| 200 μM | (% of P + 2) | 3.0 | 3.4 | 3.6 | 4.3 | 3.6 |
| | (% of P + 1) | 91.7 | 93.8 | 93.7 | 93.0 | 94.2 |
| 500 μM | (% of P + 2) | 7.5 | 9.6 | 11.1 | 11.2 | 11.5 |
| | (% of P + 1) | 88.8 | 88.4 | 86.9 | 87.0 | 87.0 |
| 1 mM | (% of P + 2) | 16.7 | 23.7 | 22.1 | 21.9 | 21.7 |
| | (% of P + 1) | 80.6 | 73.7 | 75.9 | 76.3 | 76.4 |

| | Incorporaton % of P + 1 | | | | |
|---|---|---|---|---|---|
| L-Cys-dAMP | at time points (min) | | | | |
| (compound 2) | 10 | 20 | 30 | 60 | 120 |
| 100 μM | 7.7 | 7.9 | 8.5 | 9.3 | 9.1 |
| 200 μM | 13.7 | 15.6 | 16.0 | 15.8 | 18.1 |
| 500 μM | 27.7 | 29.6 | 38.3 | 38.4 | 41.5 |
| | Incorporaton % of P + 1 | | | | |
| Tau-dAMP | at time points (min) | | | | |
| (compound 1) | 10 | 20 | 30 | 60 | 120 |
| 100 μM | 3.5 | 4.0 | 4.8 | 6.3 | 7.2 |
| 200 μM | 3.1 | 5.7 | 4.6 | 5.1 | 4.3 |
| 500 μM | 12.8 | 16.6 | 19.8 | 20.1 | 20.2 |
| 1 mM | 26.6 | 41.0 | 55.1 | 54.7 | 55.7 |

| | Incorporaton % of P + 1 | | | | |
|---|---|---|---|---|---|
| O-phospho-L-Ser-dAMP | at time points (min) | | | | |
| (compound 5) | 10 | 20 | 30 | 60 | 120 |
| 100 μM | 3.1 | 3.2 | 4.6 | 5.1 | 5.2 |
| 200 μM | 3.7 | 4.6 | 5.8 | 6.6 | 9.5 |

### Example 3

### Elongation Experiments (Using HIV-1 RT)

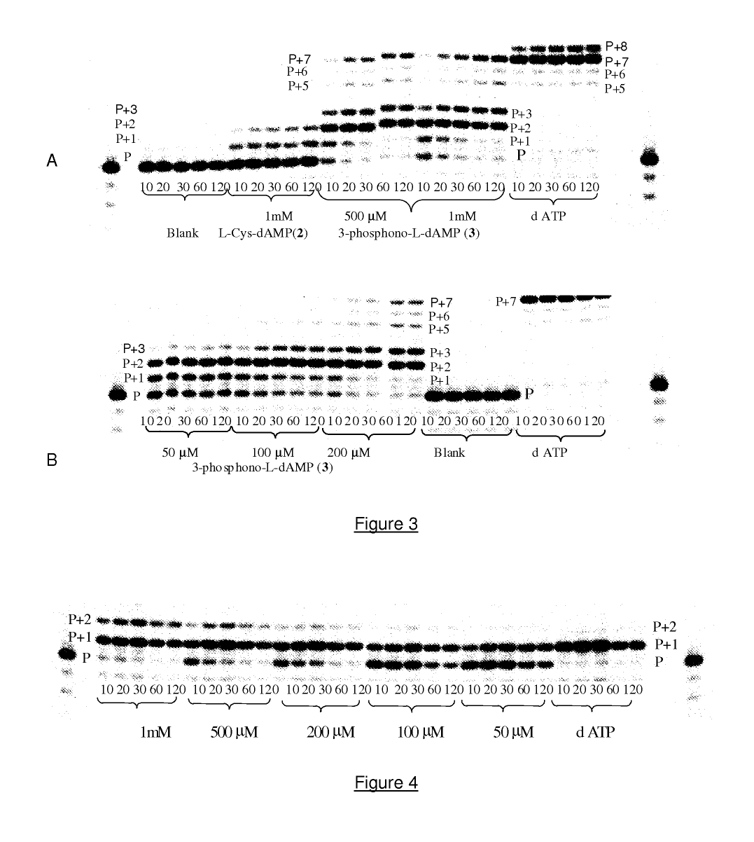

In order to further investigate the potentiality of the leaving groups of this invention, L-Cys-dAMP (2) and 3-phosphono-L-Ala-dAMP (3) were tested in the template dependent incorporation of more than one phosphoramidate nucleotides by HIV-1 RT (FIG. 4). For this purpose, Primer P₁ and template T₂ containing an overhang of seven thymidine residues and four nucleobases (GGAC) at the 3′-end were used.

Gel electrophoresis experiments showed that L-Cys-dAMP (2) only extended a primer with one and two adenine nucleobases (P+1 and P+2 products). At the same time, 3-phosphono-L-Ala-dAMP (3) provided encouraging results. After 60 minutes of the polymerase reaction, 3-phosphono-L-Ala-dAMP (3) incorporated P+7 (23.8%) at 1 mM, P+7 (15.9%) at 500 μM, and P+7 (7.1%) at 200 μM, respectively. The existed P+2 and P+3 products suggest that the incorporation by 3-phosphono-L-Ala-dAMP (3) is less active than the natural dATP. Nevertheless, 3-phosphono-L-Ala-dAMP (3) did not result in any misincorporation (P+8). Additionally, with the lower concentration of 50 μM and 100 μM, 3-phosphono-L-Ala-dAMP (3) also only extended a primer with two and three adenine nucleobases (P+2 and P+3 products).

### Example 4

### Kinetic Experiments

The efficiency of incorporation by HIV-1 RT of 3-phosphono-L-Ala-dAMP (3) was investigated by determination of the kinetic parameters Kₘ and Vₘₐₓ. The steady-state kinetics assay was carried out as follows. The reaction was started by adding HIV-1 RT to P₁-T₁ complex, buffer, 3-phosphono-L-Ala-dAMP (3) and dATP. The final mixture (20 μL) contained 0.025 U/μL HIV-1 RT, buffer, 125 nM primer-template complex, and various concentrations 3-phosphono-L-Ala-dAMP and dATP. The range of concentrations for phosphoamidates was optimized according to a Kₘ value for the incorporation of an individual nucleotide. In the case of HIV-1 RT, reaction mixtures containing the enzyme in concentration (0.025 U/μL) to attain 5-25% conversion and appropriate substrate concentration (0.5-100 μM used for 3-phosphono-L-Ala-dAMP (3) and 0.1-10 μM used for dATP) were incubated at 37° C. and run for 8-10 different time intervals (2-20 minutes). The incorporation velocities were calculated based on the percentage of single-nucleotide extension product (P+1 band). The kinetic parameters (Vₘₐₓ and Kₘ) were determined by plotting V (nM/min) versus substrate concentration (μM) and fitting the data point to a nonlinear Michaelis-Menten regression using GraphPad Prism software.

Steady-state kinetics analysis (Table 2) of the incorporation of 3-phosphono-L-Ala-dAMP (3) indicated that, although Kₘ for 3-phosphono-L-Ala-dAMP (3) is 15-fold higher than the natural substrate dATP, the measured Vₘₐₓ is only 1.7-fold lower. These data indicate efficient nucleophilic displacement of the 3-phosphono-L-Alanine when the relative phosphoramidate is bound at the active site.

TABLE 2
steady-state Kinetics of single nucleotide
incorporation by HIV-1 RT

| Substrate | Vₘₐₓ [nM/min] | Kₘ [μM] | Vₘₐₓ/Kₘ (×10³) |
|---|---|---|---|
| dATP | 32.27 ± 2.32 | 0.93 ± 0.26 | 34.7 |
| 3-phosphono-L- | 19.21 ± 1.58 | 14.31 ± 3.78 | 1.34 |
| Ala-dAMP (3) | | | |

### Example 5

### Single Nucleotide Incorporation Using Tag DNA Polymerase

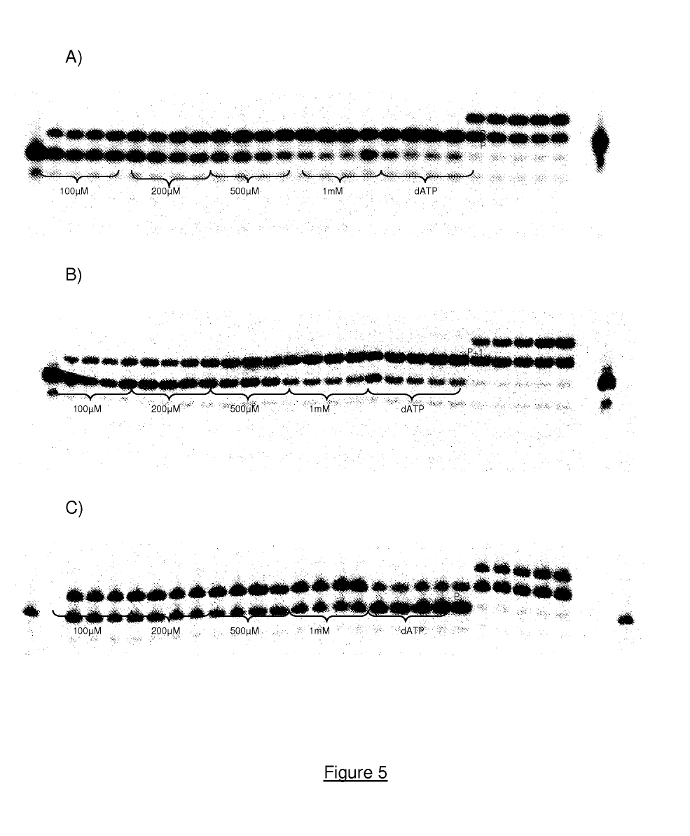

3-phosphono-L-Ala-dAMP (3) was tested as a substrate for Taq DNA polymerase (data in FIG. 5). Incorporation and primer extension by Taq DNA polymerase were observed less active than HIV-1 RT. The selectivity for HIV-1 RT directs the potential of the leaving group of compound 3 for the design of reverse transcriptase inhibitors as potential anti-HIV agents.

Overview of the primer-template complexes used in the DNA
polymerase reactions. Bold letters indicate the template
overhang in the hybridized primer-template duplex.
Single nucleotide incorporation and Kinetic experiments

| | | |
|---|---|---|
| SEQ ID NO: 1 | P1 | 5′-CAGGAAACAGCTATGAC-3′ |
| SEQ ID NO: 2 | T1 | 3′-GTCCTTTGTCGATACTGTCCC-5′ |
| [symbol] | | |

[symbol]

indicates data missing or illegible when filed

### Example 6

### Synthesis of Phosphoramidate Analogues

Compounds 6-8, structurally shown below have been prepared for evaluation as potential substrates for HIV-1 RT.

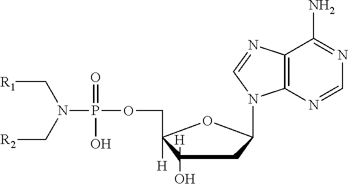

| Cpd | R₁ | R₂ |
|---|---|---|
| 6 | PO₃H₂ | PO₃H₂ |
| 7 | CH₂ PO₃H₂ | CH₂ PO₃H₂ |
| 8 | COOH | PO₃H₂ |

The synthetic route for compounds 6 and 7 is shown in the following scheme 8, the reaction conditions being: step (1) DCC, 1,4-dioxane/DMF, 80° C.; step (2) TMSI, 0° C., Et₃N, CH₂Cl₂

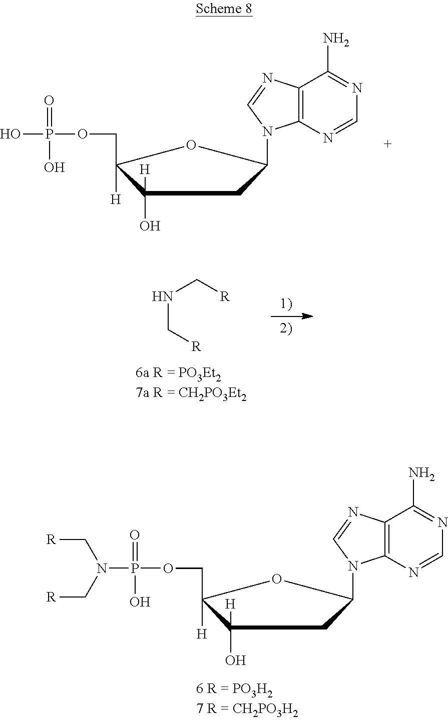

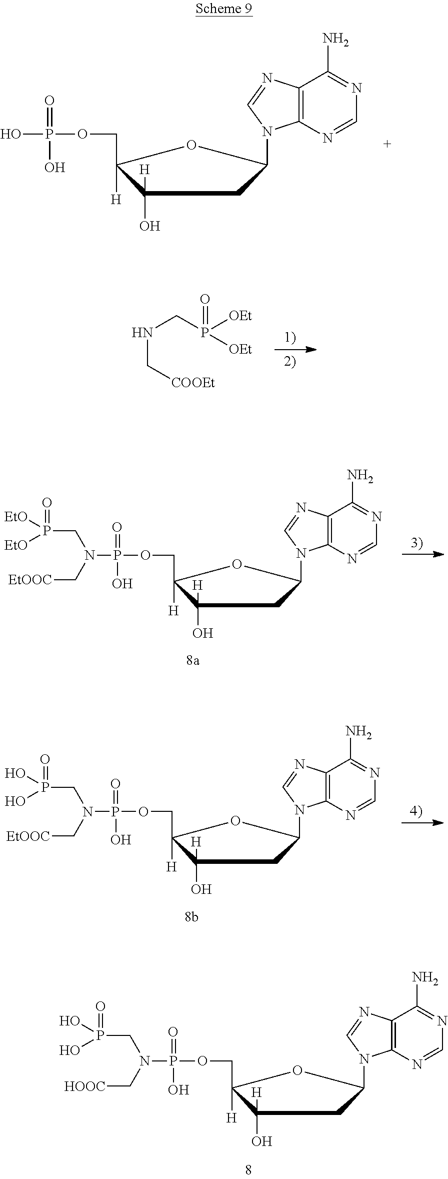

The synthetic route for compound 8 is shown in the above scheme 9, the reaction conditions being: step (1) DCC, 1,4-dioxane/DMF, 80° C.; step (2) TMSI, 0° C., Et₃N, CH₂Cl₂; step (3) 0.4M NaOH (MeOH/H₂O).

### Synthesis of 2′-deoxyadenosine-5′-[tetraethyl iminobis(methane phosphonate)] phosphoramidate (compound 6a)

The general procedure was applied using 2′-deoxyadenosine-5′-monophosphate (100 mg, 0.30 mmole) and tetraethyl iminobis(methane phosphonate) (666 mg, 2.1 mmoles), and DCC (436 mg, 2.11 mmoles) as the coupling reagent. After purification obtained white solid product (138 mg, 73% yield) which was characterized as follows:

¹H NMR (300 MHz, D₂O): δ 8.39 (s, 1H, H₈), 8.18 (s, 1H, H₂), 6.42 (t, 1H, J=5.67 Hz, H_{1′}), 4.65 (m, 1H, H_{3′}), 4.17 (m, 1H, H_{4′}), 4.01 (m, 8H, CH₂CH_{3′}), 3.89 (m, 2H, H_{5′}), 3.55 (m, 4H, CH₂PO₃CH₂CH₃), 2.81 (m, 1H, H_{2′a}), 2.55 (m, 1H, H_{2′b}), and 1.93 (m, 12H, CH₂CH₃) ppm;

¹³C NMR (125 MHz, D₂O): δ 155.3, 152.5, 148.6, 139.7, 115.3, 85.8, 83.4, 71.2, 64.1, 63.3, 42.3, 38.4, and 15.3 ppm;

³¹P NMR (121 MHz, D₂O): δ 5.58, 26.98 ppm; and

MS: calculated for C₂₀H₂₆N₆O₁₁P₃ 629.2. found: 628.8.

### Synthesis of 2′-deoxyadenosine-5′-[iminobis(methanephosphonate)] phosphoramidate (6)

To a solution of compound (6a) (100 mg, 0.158 mmole) and Et₃N (444 μL, 3.16 mmole) in 10 ml dry CH₂Cl₂ was added iodotrimethylsilane (348 μL, 2.528 mmoles) at 0° C. under argon, then reaction was stirred at room temperature for 6 hours. The reaction was quenched with 1M TEAB solution, the mixture was concentrated in vacuo, and the residue was purified by chromatography on a silica gel column (CHCl₃:MeOH 5:1, CHCl₃:MeOH: H2O 5:2:0.25, CHCl₃:MeOH: H2O 5:3:0.25, CHCl₃:MeOH: H2O 5:3:0.5, CHCl₃:MeOH:H2O 5:3:0.5, CHCl₃:MeOH:H2O 5:4:1) to give a white solid product (138 mg, 73% yield) which was characterized as follows:

¹H NMR (300 MHz, D₂O): δ 8.48 (s, 1H, H₈), 8.27 (s, 1H, H₂), 6.41 (t, 1H, J=6 Hz, H_{1′}), 4.70 (m, 1H, H_{3′}), 4.20 (m, 1H, H_{4′}), 4.01 (m, 2H, H_{5′}), 3.12 (m, 4H, CH₂PO₃H₂), 2.77 (m, 1H, H_{2′a}), and 2.55 (m, 1H, H_{2′b}) ppm;

¹³C NMR (125 MHz, D₂O): δ 154.5, 151.4, 148.2, 139.8, 118.2, 85.6, 83.5, 70.9, 64.3, 46.2, and 38.8 ppm;

³¹P NMR (121 MHz, D₂O): δ 10.09, 19.50 ppm; and

HRMS: calculated for C₁₂H₂₀N₆O₁₁P₃ 517.04082. found: 517.0417

### Synthesis of 2′-deoxyadenosine-5′-[tetraethyl iminobis(ethyl phosphonate)] phosphoramidate (7a)

The general procedure was applied using 2′-deoxyadenosine-5′-monophosphate (100 mg, 0.30 mmoles), tetraethyl iminobis(ethyl phosphonate) (725 mg, 2.1 mmoles), DCC (436 mg, 2.11 mmoles) as the coupling reagent. After purification was obtained a white solid product (105 mg, 53% yield) which was characterized as follows:

¹H NMR (300 MHz, D₂O): δ 8.50 (s, 1H, H₈), 8.21 (s, 1H, H₂), 6.49 (m, 1H, H_{1′}), 4.67 (m, 1H, H_{3′}), 4.02 (m, 9H, OCH₂CH_{3′}+H_{4′}), 3.65 (m, 2H, H_{5′}), 2.79 (m, 1H, H_{2′a}), 2.48 (m, 1H, H_{2′b}), 2.17 (m, 4H, NCH₂CH₂), 1.97 (m, 2H, NCH₂CH₂), 1.88 (m, 2H, NCH₂CH₂), and 1.32 (m, 12H, CH₂CH₃) ppm;

¹³C NMR (125 MHz, D₂O): δ 153.2, 152.4, 148.5, 118.3, 86.2, 83.4, 70.3, 63.5, 62.8, 39.8, 24.5, and 15.3 ppm;

³¹P NMR (121 MHz, D₂O): δ 7.28, 28.61 ppm; and

MS: calculated for C₂₂H₄₁N₆O₁₁P₃ 658.2. found: 658.7[M+H]⁺

### Synthesis of 2′-deoxyadenosine-5′-[iminobis(ethyl phosphonate)] phosphoramidate (7)

The general procedure (was applied using 2′-deoxyadenosine-5′-[tetraethyl iminobis(ethyl phosphonate)] phosphoramidate (80 mg, 0.12 mmole), Et₃N (337 μL, 2.4 mmoles) and iodotrimethylsilane (262 μL, 1.92 mmoles), after purification compound 7 was obtained as a white solid (42 mg, 64% yield) which was characterized as follows:

¹H NMR (300 MHz, D₂O): δ 8.47 (s, 1H, H₈), 8.28 (s, 1H, H₂), 6.45 (m, 1H, H_{1′}), 4.67 (m, 1H, H_{3′}), 4.19 (m, 1H, H_{4′}), 3.89 (m, 2H, H_{5′}), 3.10 (m, 4H, NCH₂CH₂), 2.74 (m, 1H, H_{2′a}), 2.57 (m, 1H, H_{2′b}), and 1.76 (m, 4H, NCH₂CH₂) ppm;

¹³C NMR (125 MHz, D₂O): δ 154.7, 151.8, 148.2, 139.7, 118.3, 85.6, 83.5, 70.9, 60.1, 42.9, 38.9, and 25.4 ppm;

³¹P NMR (121 MHz, D₂O): δ 8.86, 22.58 ppm; and

HRMS: calculated for C₁₄H₂₄N₆O₁₁P₃ 545.07212. found: 545.0712

### Synthesis of 2′-deoxyadenosine-5′-[(diethoxyl phosphinyl methyl) glycine ethyl ester] phosphoramidate (8a)

The general procedure was applied using 2′-deoxyadenosine-5′-monophosphate (100 mg, 0.30 mmole), N-[(diethoxy phosphinyl)methyl]glycine ethyl ester (532 mg, 2.1 mmoles), and DCC (436 mg, 2.11 mmoles) as the coupling reagent. After purification was obtained a white solid product (144 mg, 88% yield) which was characterized as follows:

¹H NMR (300 MHz, D₂O): δ 8.30 (s, 1H, H₈), 8.05 (s, 1H, H₂), 6.32 (m, 1H, H_{1′}), 4.67 (m, 1H, H_{3′}), 4.14 (m, 1H, H_{4′}), 3.94 (m, 8H, CH₂CH₃+H_{5′}), 3.69 (m, 2H, NCH₂COOEt), 3.37 (m, 2H, NCH₂PO₃Et₂), 2.79 (m, 1H, H_{2′a}), 2.54 (m, 1H, H_{2′b}), and 1.11 (m, 12H, CH₂CH₃) ppm;

¹³C NMR (125 MHz, D₂O): δ 172.6, 155.0, 152.1, 148.6, 139.9, 118.4, 86.0, 83.6, 71.5, 64.3, 63.5, 61.7, 48.8, 42.6, 38.6, 15.5, and 13.1 ppm;

³¹P NMR (121 MHz, D₂O): δ 6.68, 26.85 ppm; and

MS: calculated for C₁₉H₃₂N₆O₁₀P₂ 565.2. found: 564.

### Synthesis of 2′-deoxyadenosine-5′-[(phosphonomethyl)glycine ethyl ester] phosphoramidate (compound 8b)

The general procedure was applied using 2′-deoxyadenosine-5′-[(phosphonomethyl)glycine ethyl ester] phosphoramidate (100 mg, 0.177 mmole), Et₃N (249 μL, 1.77 mmole) and iodotrimethylsilane (193 μL, 1.42 mmoles), after purification achieving compound 8a as a white solid (69 mg, 77% yield) which was characterized as follows:

¹H NMR (300 MHz, D₂O): δ 8.40 (s, 1H, H₈), 8.15 (s, 1H, H₂), 6.41 (t, 1H, J=6.42 Hz, H_{1′}), 4.67 (m, 1H, H_{3′}), 4.17 (m, 1H, H_{4′}), 3.99 (m, 4H, CH₂CH₃+H_{8′}), 3.49 (m, 2H, NCH₂COOEt), 3.35 (m, 2H, NCH₂PO₃Et₂), 2.76 (m, 1H, H_{2′a}), 2.52 (m, 1H, H_{2b}), and 1.06 (m, 3H, CH₂CH₃) ppm;

¹³C NMR (125 MHz, D₂O): δ 173.2, 155.2, 152.2, 148.4, 139.7, 118.3, 85.8, 83.4, 71.2, 64.0, 61.3, 45.0, 43.0, 38.7, and 12.9 ppm;

³¹P NMR (121 MHz, D₂O): δ 8.12, 18.50 ppm;

MS: calculated for C₁₈H₂₄N₆O₁₀P₂ 509.1. found: 508.7

### Synthesis of 2′-deoxyadenosine-5′-[(diethoxyl phosphinyl methyl)glycine] phosphoramidate (8) The general procedure was applied using 2′-deoxyadenosine-5′-[(phosphonomethyl)glycine ethyl ester] phosphoramidate (60 mg, 0.118 mmole) and 0.4 M sodium hydroxide solution in MeOH:H₂O (4:1) (2 mL), achieving a white solid product (45 mg, 80% yield) which was characterized as follows:

¹H NMR (300 MHz, D₂O): δ 8.53 (s, 1H, H₈), 8.24 (s, 1H, H₂), 6.51 (t, 1H, J=7 Hz, H_{1′}), 4.67 (m, 1H, H_{3′}), 4.25 (m, 1H, H_{4′}), 4.10 (m, 1H, H_{5′a}), 4.02 (m, 1H, H_{5′b}), 3.79 (d, 2H, J=11.5 Hz, CH₂COOH), 3.24 (t, 2H, J=9 Hz, CH₂PO₃H₂), 2.82 (m, 1H, H₂ₐ), and 2.55 (m, 1H, H_{2′b}) ppm;

¹³C NMR (125 MHz, D₂O): δ 178.9, 155.2, 152.3, 148.3, 139.6, 118.2, 85.8, 83.2, 70.8, 63.6, 50.5, 45.1, and 38.6 ppm;

³¹P NMR (121 MHz, D₂O): δ 8.63, 19.20 ppm;

HRMS: calculated for C₁₃H₂₀N₆O₁₀P₂ 481.06432. found 481.0640.

### Example 7

### Biological Evaluation of Phosphoramidates (Compounds 6-8)

The ability of HIV-1 reverse transcriptase to incorporate phosphoramidate analogues 2-6 was analysed by gel-based single-nucleotide-incorporation assays using the primer-template complex P₁T₁. Using the same incorporation conditions as in the previous example, compound 6, 7 and 8 showed efficient incorporation with 90%, 83.2% and 70.8% conversion within 60 minutes with 500 μM substrate concentration as shown in FIGS. 6-a, 6-b and 6-c respectively.

In order to investigate the strand elongation capacity of compound 2, a template dependent incorporation of more than one nucleotide experiment was carried out. In this experiment HIV-1 RT and P1T2 duplex, where seven thymidine bases overhang of the template is flanked by four non-thymidine units at the 3′-end were used. A range of concentration of the building block was incubated with the primer-template complex and 0.025 U/μL of enzyme at the appropriate temperature, samples were quenched after 15, 30, 60, 90 and 120 minutes respectively and analyzed by 20% polyacrylamide gel electrophoresis. The elongation data are provided in the table below.

Compounds 6, 7 and 8 in the elongation of P1 directed by template
T2: % of P + n product after a 120 minutes reaction

| Product | % product ^{a} Cpd 6 | % product ^{a} Cpd 7 | % product ^{a} Cpd 8 |
|---|---|---|---|
| P + 7 | 0 | 0 | 1.2 |
| P + 6 | 0 | 0 | 0.6 |
| P + 5 | 0 | 0 | 1.2 |
| P + 4 | traces | traces | traces |
| P + 3 | 10.8 | 4.9 | 7.9 |
| P + 2 | 53.1 | 35.4 | 37.7 |
| P + 1 | 8.6 | 15.2 | 11.8 |

^{a} percentage of the total amount of radio-nucleotides in the mixture

1-30. (canceled)

31. Modified nucleosides represented by the structural formula (A): 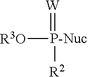
wherein
Nuc is a natural nucleoside or a nucleoside analogue;
R³ is selected from the group consisting of H, C₁₋₆ alkyl, C₃₋₆ cycloalkyl, aryl-C₁₋₆ alkyl, C₁-C₆ acyloxymethylene, C₁-C₆ alkoxycarbonyloxymethylene and 2-cyanoethyl, wherein said C₁₋₆ alkyl, C₃₋₆ cycloalkyl, C₁₋₆ acyloxymethylene, C₁₋₆ alkoxycarbonyloxymethylene or aryl-C₁₋₆ alkyl is optionally substituted with one or more, preferably 1, 2 or 3, substituents independently selected from the group consisting of halogen, OH, C₁₋₆ alkoxy, trifluoromethyl, trifluoromethoxy, nitro, cyano and amino;
W is O or S;
R² is represented by the structural formula (II): 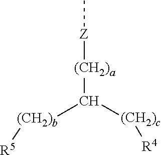
wherein
dotted lines represent the point of attachment of Z to the phosphorous atom P of the structural formula (A);
Z is selected from the group consisting of O, S, NH and NR⁷; and
R⁷ is selected from the group consisting of C₁₋₆ alkyl, phenyl, benzyl and cyclohexyl;
a is 0 or 1;
b is 0, 1 or 2;
c is 0, 1 or 2 or 3;
R⁵ is selected from the group consisting of hydrogen; aryl; imidazolyl; P(O)(OH)₂; O—P(O)(OH)₂; S(O)₂(OH); O—S(O)₂(OH); and COOR⁶, wherein R⁶ is hydrogen or C₁₋₆ alkyl;
R⁴ is selected from the group consisting of P(O)(OH)₂, O—P(O)(OH)₂, S(O)₂(OH) and O—S(O)₂(OH); or, provided that Z is NH, a=b=0, c is 1 and R⁵ is COOH, R⁴ is selected from the group consisting of C₆H₅—OP(O)(OH)₂ wherein said C₆H₅ (phenyl) is substituted with fluoromethyl or difluoromethyl; C₆H₅—CHXP(O)(OH)₂; C₆H₅-QS(O)₂(CH═CH₂); C₆H₅-QV wherein said C₆H₅ (phenyl) is substituted with oxiran-2-yl or CH═CH₂; C₆H₅—C(═CH₂)V; CHXV and C(═CH₂)V;
X is chloro or bromo;
Q is a linking moiety selected from the group consisting of O, CH₂, (CH₂)₂ and CF₂;
V is selected from the group consisting of P(O)(OH)₂, S(O)₂(OH), SO₂NH₂, SO₂CH₃ and SO₂CF₃;
or R² is represented by the structural formula (V): 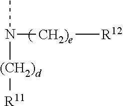
wherein
dotted lines represent the point of attachment of N to the phosphorous atom of formula (A);
d is 0, 1, 2, 3 or 4;
e is 0, 1, 2, or 3;
R¹² is selected from the group consisting of P(O)(OH)₂, O—P(O)(OH)₂, S(O)₂(OH) or O—S(O)₂(OH);
R¹² is selected from the group consisting of hydrogen; aryl; imidazolyl; P(O)(OH)₂; O—P(O)(OH)₂; S(O)₂(OH); O—S(O)₂(OH); and COOR⁶, wherein R⁶ is hydrogen or C₁₋₆ alkyl;
and stereoisomers, pharmaceutically acceptable salts and pro-drugs thereof,
provided that said phosphate-modified nucleoside is not one wherein R² is represented by the structural formula (V) and wherein d=0, e=0, R¹² is hydrogen or aryl, and R¹¹ is P(O)(OH)₂;
provided that said phosphate-modified nucleoside is not one wherein R² is represented by the structural formula (II) wherein Z is O, a=0, b=0, R⁵ is aryl, c=0 and R⁴ is P(O)(OH)₂;
provided that said phosphate-modified nucleoside is not one wherein R² is represented by the structural formula (II) wherein b and c are both 0; or wherein b and c are both 0 when Z is 0; and
provided that said phosphate-modified nucleoside is not one wherein R² is represented by the structural formula (V) wherein d is 0, or wherein d is 0 and Z is 0.

32. Modified nucleotides represented by the structural formula (I): 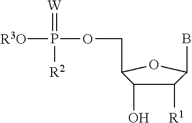
wherein
B is a pyrimidine or purine base, or an analogue thereof, optionally substituted with one or two substituents independently selected from the group consisting of halogen, hydroxyl, sulfhydryl, methyl, ethyl, isopropyl, amino, methylamino, ethylamino, trifluoromethyl and cyano;
R¹ is H or OH;
R³ is selected from the group consisting of H, C₁₋₆ alkyl, C₃₋₆ cycloalkyl, aryl-C₁₋₆ alkyl, C₁₋₆ acyloxymethylene, C₁₋₆ alkoxycarbonyloxymethylene and 2-cyanoethyl, wherein said C₁₋₆ alkyl, C₃₋₆ cycloalkyl, C₁₋₆ acyloxymethylene, C₁₋₆ alkoxycarbonyloxymethylene or aryl-C₁₋₆ alkyl is optionally substituted with one or more, preferably 1, 2 or 3, substituents independently selected from the group consisting of halogen, OH, C₁₋₆ alkoxy, trifluoromethyl, trifluoromethoxy, nitro, cyano and amino;
W is O or S; and
R² is represented by the structural formula (II): 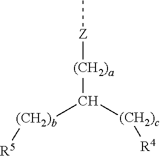
wherein
dotted lines represent the point of attachment of Z to the phosphorous atom P of the structural formula (I);
Z is selected from the group consisting of O; S; NH and NR';
R⁷ is selected from the group consisting of C₁₋₆ alkyl, phenyl, benzyl and cyclohexyl,
a is 0 or 1;
b is 0, 1 or 2;
c is 0, 1 or 2 or 3;
R⁵ is selected from the group consisting of hydrogen; aryl; imidazolyl; P(O)(OH)₂; O—P(O)(OH)₂;
S(O)₂(OH); O—S(O)₂(OH); and COOR⁶, wherein R⁶ is hydrogen or C₁₋₆ alkyl;
R⁴ is selected from the group consisting of P(O)(OH)₂, O—P(O)(OH)₂, S(O)₂(OH) and O—S(O)₂(OH); or, provided that Z is NH, a=b=0, c is 1 and R⁵ is COOH, R⁴ is selected from the group consisting of C₆H₅—OP(O)(OH)₂ wherein said C₆H₅ (phenyl) is substituted with fluoromethyl or difluoromethyl; C₆H₅—CHXP(O)(OH)₂; C₆H₅-QS(O)₂(CH═CH₂); C₆H₅-QV wherein said C₆H₅ (phenyl) is substituted with oxiran-2-yl or CH═CH₂; C₆H₅—C(═CH₂)V; CHXV and C(═CH₂)V;
X is chloro or bromo;
Q is a linking moiety selected from the group consisting of O, CH₂, (CH₂)₂ and CF₂;
V is selected from the group consisting of P(O)(OH)₂, S(O)₂(OH), SO₂NH₂, SO₂CH₃ and SO₂CF₃;
or R² is represented by the structural formula (V): 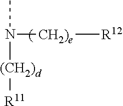
wherein
dotted lines represent the point of attachment of N to the phosphorous atom of formula (I);
d is 0, 1, 2, or 3;
e is 0, 1, 2, or 3;
R¹¹ is selected from the group consisting of P(O)(OH)₂, O—P(O)(OH)₂, S(O)₂(OH) and O—S(O)₂(OH);
R¹² is selected from the group consisting of aryl; imidazolyl; P(O)(OH)₂; O—P(O)(OH)₂; S(O)₂(OH); O—S(O)₂(OH); and COOR⁶, wherein R⁶ is hydrogen or C₁₋₆ alkyl;
and stereoisomers, pharmaceutically acceptable salts and pro-drugs thereof,
provided that said phosphate-modified nucleoside is not one wherein R² is represented by the structural formula (V) and wherein d=0, e=0, R¹² is hydrogen or aryl, and R¹¹ is P(O)(OH)₂;
provided that said phosphate-modified nucleoside is not one wherein R² is represented by the structural formula (II) wherein Z is O, a=0, b=0, R⁵ is aryl, c=0 and R⁴ is P(O)(OH)₂;
provided that said phosphate-modified nucleoside is not one wherein R² is represented by the structural formula (II) wherein b and c are both 0, or wherein b and c are both 0 when Z is O; and
provided that said phosphate-modified nucleoside is not one wherein R² is represented by the structural formula (V) wherein d is 0, or wherein d is 0 and Z is 0.

33. The phosphate-modified nucleoside of claim 31 wherein R² is represented by the structural formula (II) and wherein a is 0 or 1.

34. The phosphate-modified nucleoside of claim 32 wherein R² is represented by the structural formula (II) and wherein a is 0 or 1.

35. The phosphate-modified nucleoside of claim 31 wherein R² is represented by the structural formula (II) and wherein c is 1.

36. The phosphate-modified nucleoside of claim 32 wherein R² is represented by the structural formula (II) and wherein c is 1.

37. The phosphate-modified nucleoside of claim 31 wherein R² is represented by the structural formula (II) and wherein b is 0.

38. The phosphate-modified nucleoside of claim 32 wherein R² is represented by the structural formula (II) and wherein b is 0.

39. The phosphate-modified nucleoside of claim 31 wherein R⁵ is COOH.

40. The phosphate-modified nucleoside of claim 32 wherein R⁵ is COOH.

41. The phosphate-modified nucleoside of claim 31 wherein R² is represented by the structural formula (II) and wherein R⁴ is P(O)(OH)₂.

42. The phosphate-modified nucleoside of claim 32 wherein R² is represented by the structural formula (II) and wherein R⁴ is P(O)(OH)₂.

43. The phosphate-modified nucleoside of claim 31 wherein R² is represented by the structural formula (V) and wherein d is 1.

44. The phosphate-modified nucleoside of claim 32 wherein R² is represented by the structural formula (V) and wherein d is 1.

45. The phosphate-modified nucleoside of claim 31, wherein R² is represented by the structural formula (V) and wherein e is 0 or 1.

46. The phosphate-modified nucleoside of claim 32, wherein R² is represented by the structural formula (V) and wherein e is 0 or 1.

47. The phosphate-modified nucleoside of claim 31 wherein R³ is hydrogen.

48. The phosphate-modified nucleoside of claim 32 wherein R³ is hydrogen.

49. A phosphate-modified nucleoside being selected from the group consisting of 2′-deoxyadenosine-5′-(3-phosphono-L-alanine)phosphoramidate (3-phosphono-L-Ala-dAMP); 2′-deoxyguanosine-5′-(3-phosphono-L-alanine)phosphoramidate (3-phospho-no-L-Ala-dGMP); 2′-deoxy-thymidine-5′-(3-phosphono-L-alanine)phosphoramidate (3-phosphono-L-Ala-dTMP); 2′-deoxyuridine-5′-(3-phosphono-L-alanine)phosphor-amidate (3-phosphono-L-Ala-dUMP); 2′-deoxycytidine-5′-(3-phosphono-L-alanine)-phosphoramidate (3-phosphono-L-Ala-dCMP); adenosine-5′-(3-phosphono-L-alanine)phosphoramidate (3-phosphono-L-Ala-AMP); guanosine-5′-(3-phosphono-L-alanine)phosphoramidate (3-phosphono-L-Ala-GMP); 5-methyluridine-5′-(3-phosphono-L-alanine)phosphoramidate (3-phosphono-L-Ala-m5 uMP); uridine-5′-(3-phosphono-L-alanine)phosphoramidate (3-phosphono-L-Ala-UMP); and cytidine-5′-(3-phosphono-L-alanine)phosphoramidate (3-phosphono-L-Ala-CMP).

50. The phosphate-modified nucleoside of claim 31, wherein said pyrimidine or purine base is represented by the structural formula (C): 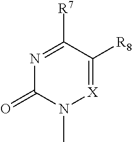
wherein
R⁷ is selected from the group consisting of OH, SH, NH₂, NHCH₃ and NHC₂H₃;
R⁸ is selected from the group consisting of hydrogen, methyl, ethyl, isopropyl, amino, ethylamino, trifluoromethyl, cyano and halogen; and
X is CH or N.

51. The phosphate-modified nucleoside of claim 32, wherein said pyrimidine or purine base is represented by the structural formula (C): 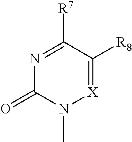
wherein
R⁷ is selected from the group consisting of OH, SH, NH₂, NHCH₃ and NHC₂H₃;
R⁸ is selected from the group consisting of hydrogen, methyl, ethyl, isopropyl, amino, ethylamino, trifluoromethyl, cyano and halogen; and
X is CH or N.

52. The phosphate-modified nucleoside of claim 31, wherein said pyrimidine or purine base is represented by the structural formula (D): 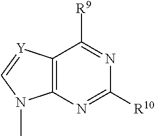
wherein
R⁹ is selected from the group consisting of H, OH, SH, NH₂, and NHCH₃;
R¹⁰ is selected from the group consisting of hydrogen, methyl, ethyl, hydroxyl, amino and halogen; and
Y is CH or N.

53. The phosphate-modified nucleoside of claim 32, wherein said pyrimidine or purine base is represented by the structural formula (D): 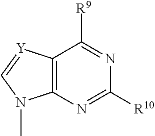
wherein
R⁹ is selected from the group consisting of H, OH, SH, NH₂, and NHCH₃;
R¹⁰ is selected from the group consisting of hydrogen, methyl, ethyl, hydroxyl, amino and halogen; and
Y is CH or N.

54. A substrate for a DNA/RNA polymerase comprising the phosphate-modified nucleosides of claim 31.

55. A substrate for a DNA/RNA polymerase comprising the phosphate-modified nucleosides of claim 32.

56. The substrate of claim 54, wherein said polymerase is from a micro-organism or from bacterial or viral origin.

57. The substrate of claim 55, wherein said polymerase is from a micro-organism or from bacterial or viral origin.

58. The substrate of claim 54, wherein the polymerase is selected from the group consisting of Therminator DNA polymerase, KF (exo⁻) DNA polymerase and Reverse Transcriptase.

59. The substrate of claim 55, wherein the polymerase is selected from the group consisting of Therminator DNA polymerase, KF (exo⁻) DNA polymerase and Reverse Transcriptase.

60. The substrate of claim 54 for building at least one nucleotide in a growing DNA- or RNA-strand.

61. The substrate of claim 55 for building at least one nucleotide in a growing DNA- or RNA-strand.

62. A method for sustaining growth, survival or proliferation of a living organism selected from the group consisting of a virus, a bacterium, an archaeon and an eukaryote, comprising the administration of the phosphate-modified nucleosides of claim 31 to said living organism.

63. The method of claim 62, wherein said eukaryote is selected from the group consisting of yeast, mold, fungus, microalga, multicellular plant and protist.

64. A composition comprising a phosphate-modified nucleoside according to claim 31, an aqueous solution and optionally one or more buffering agents, and optionally one or more nucleoside triphosphates (NTP).

65. A composition comprising a phosphate-modified nucleoside according to claim 32, an aqueous solution and optionally one or more buffering agents, and optionally one or more nucleoside triphosphates (NTP).

66. A pharmaceutical or veterinary composition comprising an anti-virally effective amount of a phosphate-modified nucleoside according to claim 31, and one or more pharmaceutically or veterinary acceptable excipients.

67. A pharmaceutical or veterinary composition comprising an anti-virally effective amount of a phosphate-modified nucleoside according to claim 32, and one or more pharmaceutically or veterinary acceptable excipients.

68. A method of prevention or treatment of a viral infection in a mammal comprising the administration, to said mammal in need thereof, of an antiviral amount of a phosphate-modified nucleoside according to claim 31, optionally in combination with one or more pharmaceutically acceptable excipients.

69. The method of claim 68, wherein said viral infection is a HIV infection.

70. A method of prevention or treatment of a viral infection in a mammal comprising the administration, to said mammal in need thereof, of an antiviral amount of a phosphate-modified nucleoside according to claim 32, optionally in combination with one or more pharmaceutically acceptable excipients.

71. The method of claim 70, wherein said viral infection is a HIV infection.


SEQUENCE LISTING (2 sequences)

SEQ ID NO: 1 - DNA, 17 bp, Artificial
  primer
caggaaacagctatgac

SEQ ID NO: 2 - DNA, 22 bp, Artificial
  primer
ggggacagtatcgacaaaggac